(19) United States
(12) Patent Application Publication
(10) Pub. No.: US 2018/0228886 A1
(43) Pub. Date: Aug. 16, 2018

(54) USE OF A LINEAR B CELL EPITOPE OF NS1 PROTEIN TO TREAT DENGUE VIRUS

(71) Applicant: University of Pittsburgh - Of the Commonwealth System of Higher Education, Pittsburgh, PA (US)

(72) Inventors: Ernesto Torres De Azeved Marques, Pittsburgh, PA (US); Eduardo Jose Moura do Nascimento, Allentown, PA (US); Guangchao Gu, Beijing (CN)

(73) Assignee: University of Pittsburgh - Of the Commonwealth System of Higher Education, Pittsburgh, PA (US)

(21) Appl. No.: 15/772,021

(22) PCT Filed: Oct. 31, 2016
(86) PCT No.: PCT/US2016/059737

§ 371 Date: Apr. 27, 2018

Related U.S. Application Data
(60) Provisional application No. 62/248,598, filed on Oct. 30, 2015.

(51) Int. Cl. A61K 39/12; C07K 14/005; G01N 33/569

(52) U.S. Cl. CPC A61K 39/12; C07K 14/005; G01N 33/56983; A61K 2039/545; C12N 2770/24123; C12N 2770/24134; C12N 2770/24122

(57) ABSTRACT

Disclosed herein are isolated flavivirus polypeptides comprising an amino acid sequence at least 75% identical to one of SEQ ID NOs: 1-11, or a nucleic acid encoding the polypeptide, or a viral like particle encoding the polypeptide, wherein the polypeptide does not comprise the amino acid sequence of a full-length flavivirus NS1 polypeptide. Methods of using these polypeptides are also disclosed, such as for preventing, treating, or determining the prognosis of a flavivirus infection.

### CROSS REFERENCE TO RELATED APPLICATIONS

This claims the benefit of U.S. Provisional Application No. 62/248,598, filed Oct. 30, 2015, which is incorporated herein by reference.

### ACKNOWLEDGMENT OF GOVERNMENT SUPPORT

This invention was made with government support under grant no. U19 AI56541 and grant no U19 AI082632 awarded by the National Institutes of Health (NIH). The government has certain rights in the invention.

### FIELD

This disclosure relates to the field of flaviviruses, specifically to methods that utilize a non-structural (NS1) epitope to determine disease severity, and methods of treating a dengue virus infection using the NS1 epitope.

### BACKGROUND

Dengue is one of the most important arthropod-borne viral infections of humans. Worldwide, over 100 million infections occur every year, including 500,000 hospitalizations, with 2.5 billion people at the risk of being infected. Endemic areas are mainly tropical and sub-tropical countries in Southeast Asia, the Pacific and the Americas (1). Dengue virus (DV), the causative agent of dengue, is a member of genus Flavivirus, Flaviviridae family, with four antigenically distinct serotypes (DV1-DV4). Infection by DV can cause a full spectrum of clinical manifestations, ranging from asymptomatic to a mild flu-like disease called dengue fever (DF). However, 5-10% of the cases progress to a severe, sometimes life threatening manifestation called dengue hemorrhagic fever (DHF), characterized by high fever, bleeding, thrombocytopenia and haemoconcentration (Peeling et al., Nat Rev Microbiol. 2010; 8(12 Suppl):S30-8). DHF is associated with multiple exposures to DV, due to the antibody dependent enhancement (ADE) caused by non-neutralizing antibodies targeting the virion generated during previous heterotypical infection (Halstead, Science. 1988; 239(4839):476-81; Halstead, Curr Opin Infect Dis. 2002; 15(5):471-6; Halstead, Adv Virus Res. 2003; 60(421-67). Infants born from mothers immune to dengue also have a high risk of developing DHF in primary infection during the first year of life, probably due to the presence of sub-neutralizing maternal antibody levels against DV (Halstead et al., Emerg Infect Dis. 2002; 8(12):1474-9). However, adult DHF cases after primary infections are well documented (Cordeiro et al., Am J Trop Med Hyg. 2007; 77(6):1128-34; Ong et al., Int J Infect Dis. 2007; 11(3):263-7; Wichmann et al., Trop Med Int Health. 2004; 9(9):1022-9) suggesting that ADE may not be the only factor associated with disease severity (Nascimento et al., PLoS One. 2009; 4(11):e7892; Nascimento et al., PLoS One. 2009; 4(8):e6782). Alternative hypothesis includes over-reactive T cells through antigenic sin (Mongkolsapaya et al., Nat Med. 2003; 9(7):921-7.), virus virulence (Diamond et al., J Virol. 2000; 74(17):7814-23.; Rico-Hesse, Adv Virus Res. 2003; 59(315-4) and host genetics (Acioli-Santos et al., Hum Immunol. 2008; 69(2):122-8; Pastor et al., Hum Immunol. 2013; 74(9):1225-30; Xavier Eurico de Alencar et al., J Trop Med. 2013; 2013(648475).

DV is a small, enveloped, positive single-strand RNA virus. The whole genome (around 11 kb) has a single open-reading frame, which encodes three structural proteins [capsid (C), membrane (M) and envelope (E) glycoproteins], and seven non-structural proteins (NS1, NS2A, NS2B, NS3, NS4A, NS4B and NS5) (Guzman et al., Nat Rev Microbiol. 2010; 8(12 Suppl):S7-16). NS1 is one of the main targets for the antibody responses after virus infection (Rothman, at Rev Immunol. 2011; 11(8):532-43). Once produced, NS1 is either secreted out of the infected cells or remains associated with cell membrane (Muller and Young, Antiviral Res. 2013; 98(2):192-208). High levels of circulating NS1, during acute phase of infection, make this protein an important biomarker for active dengue infection (Qui et al., Clin Vaccine Immunol. 2009; 16(1):88-95). A need remains for agents that can be used to treat and prevent DV, such as vaccines that include epitopes of the non-structural proteins of DV.

### SUMMARY

Compositions are disclosed herein that include a flavivirus DV3-D10 polypeptide, a nucleic acid encoding the polypeptide, and/or a viral-like particle including the polypeptide. The flavivirus can be dengue virus (DV), West Nile Virus (WNV), Yellow Fever (YF) and/or Japanese Encephalitis Virus (JEV).

Methods are disclosed herein for eliciting an immune response to a flavivirus. The method includes the administration of a composition including the a flavivirus DV3-D10 polypeptide, a nucleic acid encoding the polypeptide, and/or a viral-like particle including the polypeptide.

In some embodiments, methods are also disclosed herein for determining the prognosis of a DV infection, such as to determine disease severity. In additional embodiments, methods are also disclosed for determining the effectiveness of therapeutic agents or vaccines against the flavivirus. These methods include measuring antibodies that specifically bind a DV3-D10 polypeptide in a biological sample from a subject.

The foregoing and other objects, features, and advantages of the invention will become more apparent from the following detailed description, which proceeds with reference to the accompanying figures.

### BRIEF DESCRIPTION OF THE DRAWINGS

FIGS. 1A-1D. Hematocrit, platelet counting and liver enzymes levels on blood samples collected up to 10 days post onset of symptoms from confirmed dengue cases with varied clinical outcomes.

FIG. 2. Schematic representation of linear B cell epitope mapping strategy on DV3 NS1 protein.

FIG. 3. Peptide screening for identification of linear B cell epitope on DV3 NS1. Pooled human serum samples from DV3 cases (both primary and secondary) collected between 20 to 60 days post onset of symptoms were used to screen a library of 76 overlapping peptides (15-mers) covering the DENV3 NS1 protein sequence by ELISA. Dengue naïve serum was used as negative control. Three independent screenings were carried out, shown here is one representative screening result.

FIG. 4. Cross-reactivity between IgG against antigens from other Flavivirus and DV3-D10. Hyperimmune sera against other Flaviviruses (YF, WNV and JEV) were tested for binding to DV3-D10 by ELISA. DENV3-D10 pool (IgG positive for D10) and DENY naive serum are used as positive and negative control respectively. YFV: Yellow Fever Virus; WNV: West Nile virus; JEV: Japanese encephalitis virus.

FIGS. 5A-5D. Schematic representation of structures and surface charge distribution of the D10-homologous residues onto the NS1 proteins of DV1 and DV2. (A-B) Schematic representation of the crystallographic structures of the NS1 proteins of DV1 (PDB ID: 4OIG) (Edeling et al., Proc Natl Acad Sci USA. 2014; 111(11):4285-90) and DV2 (PDB ID: 4O6B) (Akey et al., Science. 2014; 343(6173):881-5). The regions highlighted represent the D10-homologous residues in DV1 (shown in blue on panel A) and DV2 (shown on panel B). Panels C-D show surface charge distribution of the D10-homologous residues onto the DV1 and DV2 NS1 proteins. Surface charge is represented by the corresponding electrostatic potential mapped onto the molecular surface of the D10-homologous epitope regions. Positively charged and negatively charged potential are shown in grey, with neutral regions in white. Potential interval is from −5 to +5 kJ.mol-1.e-1. Proteins are represented as a cartoon model and secondary structure elements are shown as: α-helices: spirals; β-strands: arrows; and loops and unstructured regions: coils.

FIGS. 6A-6B. IgG avidity analysis of a reference pooled samples positive for IgG anti DV3-D10. Peptide-specific ELISA was carried out with or without a urea wash using reference sample serially diluted. Then, percentage IgG avidity anti DV3-D10 and percentage of binding reduction were calculated as described on methods. Panel (A) shows three independent reference curves with and without urea wash. Panel (B) shows the antibody titers calculated from the reference curves with and without urea wash.

FIGS. 7A-7B. Initial screening for the presence of IgG anti DV3-D10 on samples from confirmed DV cases collected 20-60 days post onset of symptoms. Peptide ELISA assay was carried out and the frequency of patients positive and negative for the presence of IgG anti D10 (A) as well as antibody levels [per optical density (OD)] are shown.

FIG. 8. Comparison of IgG anti DV3 D10 titers between primary and secondary dengue infections at multiple time points post onset of symptoms. Based on the initial screening, patients positive for IgG anti DV3-D10 were selected for IgG anti DV3 D10 quantification. Levels of this antibody were analyzed based on type of infection (primary vs. secondary) only.

FIGS. 9A-9F. Comparison of IgG anti DV3 D10 titers considering both type of infection and clinical outcome. (A) shows the comparison between mild (DF) and severe disease (DFC+DHF) at multiple time points. (B-F) show the analysis taking into consideration both type of infection and clinical outcome in each time-point analyzed.

FIG. 10 Peptide ELISA assay results for the positive and negative controls. Positive control samples were used to validate the data for each plate analyzed and ranged from 0.868 to 1.369 [mean/SD: 1.072/0.122 (n=36)]. Negative controls were used to calculate assay cut-off and they ranged from 0.309 to 0.553 [man/SD: 0.491/0.078 (n=34)].

FIGS. 11A-11C. In silico analysis showing biochemical features of DV3-D10 region. ExPASy server (web.expasy.org/protscale/) was used to analyze hydrophaticity. (A): values below 0 represent hydrophilic regions on DV3 NS1 protein sequence. The grey box represent where DV3-D10 is located. (B) surface accessibility plot and (C) flexibility were analyzed using IEDB prediction resource website (tools.immuneepitope.org/bcell/) using DV3 NS1 protein sequence. For both surface accessibility and flexibility, scores above 1 represent regions accessible on the protein surface and flexible respectively. On both plots, DV3-D10 region is highlighted on the boxes.

FIG. 12. Calibration curves for the peptide ELISA to determine assay reproducibility. Reference sample for IgG anti DV3-D10 was serially diluted and used for peptide ELISA assay. A total of 18 curves were performed by two individuals in multiple days and plotted together. Four-parameters non-linear regression was applied to each curve individually. Bottom, top, Log EC50, hillslope and span were noted for each curve and used to calculate the inter-assay variances. The assay was considered reproducible if the inter-assay variances for top, LogEC50, hillslope and span were below 5%.

FIG. 13. Representative peptide ELISA data during IgG anti DV3-D10 quantification. Serum samples at multiple time points post onset of symptoms were used on peptide ELISA assay to determine their IgG anti DV3-D10 levels. Lines with circle (•), square ( ) triangle ( ) and “X” show samples with titers >1:800, between 1:800 and 1:500, between 1:500 and 1:100 and <1:100 respectively. Dashed line represents the assay cut-off. Samples in red were subjected to a second round of titration starting with at 1:400 dilution (see the Examples section).

FIG. 14. Cross-reactivity of antibodies produced in response to DV1 and DV2 infections to DV3-D10. Serum samples collected 8-60 days post onset of symptoms from primary DV1 and DV2 primary infections were used on peptide ELISA assay. DV3 primary samples were used as control for the assay. Percentage shown on top of the chart represent number of responders/total number of individuals tested based on the cut-off calculated (dashed line).

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file [Sequence_Listing, Oct. 31, 2015, size of 2,921 bytes], which is incorporated by reference herein.

### DETAILED DESCRIPTION

Dengue is caused by Dengue virus (DV), a member of genus Flavivirus with four antigenically distinct serotypes (DV1-DV4). Clinical outcome varies from a mild (DF) to a severe and sometimes fatal manifestation (DHF). NS1 plays a role on disease immunopathology and is one of the main targets of antibody responses during DV infection.

Currently there is no reliable prognostic tool to predict disease outcome in patients infected with dengue virus (DV). In addition, no vaccine has been licensed yet to prevent the development of DHF. Disclosed herein is use of an IgG biomarker of protection against development of DHF. Also disclosed are a prophylactic and therapeutic methods to prevent and/or treat a dengue virus infection.

The disclosed methods utilize the identification of IgG interacting with a linear B cell epitope present on Dengue virus NS1 protein (position 209-223, called DV3-D10) that indicates protection against disease severity caused by DV. The DV3-D10 epitope, nucleic acids encoding this polypeptide, and/or viral like particles (VLPs) including this polypeptide can be used to induce an immune response, and thus to treat and/or prevent DV infection or DHF. Moreover, the peptide 209-SWKLEKASLIEVKTC-223 (SEQ ID NO: 1) as well as its homologous region on other DV serotypes and Flaviviruses, as disclosed herein, can be used as a prophylactic means to elicit IgG responses to ultimately prevent the development of disease severity caused by DV.

Antibody levels against DV3-D10 were determined throughout the course of the disease. Primary infections have constant, low levels of IgG that specifically binds DV3-D10, whereas secondary infections have a burst of antibody production at the febrile phase of infection (1-5 days). Secondary DF cases have greater levels of DV3-D10 specific IgG also during the febrile phase, before the vasculopathy symptoms take place in DHF patients. Thus, the presence of this antibody represents a protective factor against development of DHF.

In some embodiments, the presence of the antibody can be used to distinguish subjects that will (or will not) develop DHF. In a specific non-limiting example, subjects that have an increase in antibodies that specifically bind DV3-D10, as compared to a control, can be treated less aggressively. In a particular example, these subjects can be treated at home. In another specific non-limiting example, subject that do not have an increase in antibodies that specifically bind DV3-D10, as compared to a control, can be treated more aggressively. In a particular example, these subject can be treated in a clinical setting, such as a hospital.

This epitope is present on all DV serotypes and also on other Flaviviruses, such as West Nile Virus (WNV) and Japanese Encephalitis (JEV). IgG against this novel epitope, as well as its homologous region on other DV serotypes and Flaviviruses, also can be used as: (i) a prognostic marker of protection from disease severity caused by DV; and (ii) therapeutic means to reduce disease severity caused by any DV serotype.

### Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Adjuvant: A substance or vehicle that non-specifically enhances the immune response to an antigen. Adjuvants can include a suspension of minerals (alum, aluminum hydroxide, or phosphate) on which antigen is adsorbed; or water-in-oil emulsion in which antigen solution is emulsified in mineral oil (for example, Freund's incomplete adjuvant), sometimes with the inclusion of killed mycobacteria (Freund's complete adjuvant) to further enhance antigenicity. Immunostimulatory oligonucleotides (such as those including a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants also include biological molecules, such as costimulatory molecules. Exemplary biological adjuvants include IL-2, RANTES, GM-CSF, TNF-α, IFN-γ, G-CSF, LFA-3, CD72, B7-1, B7-2, OX-40L and 41 BBL.

Administer: As used herein, administering a composition to a subject means to give, apply or bring the composition into contact with the subject. Administration can be accomplished by any of a number of routes, such as, for example, topical, oral, subcutaneous, intramuscular, intraperitoneal, intravenous, intrathecal and intradermal.

Antibody: An immunoglobulin molecule produced by B lymphoid cells with a specific amino acid sequence. Antibodies are evoked in humans or other animals by a specific antigen (immunogen). Antibodies are characterized by reacting specifically with the antigen in some demonstrable way, antibody and antigen each being defined in terms of the other. “Eliciting an antibody response” refers to the ability of an antigen or other molecule to induce the production of antibodies.

Typically, a naturally occurring immunoglobulin has heavy (H) chains and light (L) chains interconnected by disulfide bonds Immunoglobulin genes include the kappa, lambda, alpha, gamma, delta, epsilon and mu constant region genes, as well as the myriad immunoglobulin variable domain genes. There are two types of light chain, lambda (λ) and kappa (κ). There are five main heavy chain classes (or isotypes) which determine the functional activity of an antibody molecule: IgM, IgD, IgG, IgA and IgE.

Light and heavy chain variable regions contain a “framework” region interrupted by three hypervariable regions, also called “complementarity-determining regions” or “CDRs” (see, e.g., Kabat et al., Sequences of Proteins of Immunological Interest, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space. The CDRs are primarily responsible for binding to an epitope of an antigen.

A “monoclonal antibody” is an antibody produced by a single clone of B-lymphocytes or by a cell into which nucleic acid encoding the light and heavy chains of a single antibody have been transfected, or a progeny thereof. Monoclonal antibodies are produced by methods known to those of skill in the art, for instance by making hybrid antibody-forming cells from a fusion of myeloma cells with immune spleen cells. These fused cells and their progeny are termed “hybridomas.” In some examples monoclonal antibodies are isolated from a subject. Monoclonal antibodies can have conservative amino acid substitutions which have substantially no effect on antigen binding or other immunoglobulin functions. (See, for example, Harlow & Lane, Antibodies, A Laboratory Manual, 2^{nd} ed. Cold Spring Harbor Publications, New York (2013).)

Antigen: A compound, composition, or substance that can stimulate the production of antibodies or a T-cell response in an animal, including compositions that are injected or absorbed into an animal. An antigen reacts with the products of specific humoral or cellular immunity, including those induced by heterologous immunogens. In some embodiments of the disclosed compositions and methods, the antigen is an NS1 protein or epitope thereof such as DV3-D10.

Conditions sufficient to form an immune complex: Conditions which allow an antibody or antigen binding fragment thereof to bind to its cognate epitope to a detectably greater degree than, and/or to the substantial exclusion of, binding to substantially all other epitopes. Conditions sufficient to form an immune complex are dependent upon the format of the binding reaction and typically are those utilized in immunoassay protocols or those conditions encountered in vivo. See Harlow & Lane, infra, for a description of immunoassay formats and conditions. The conditions employed in the methods are “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (e.g., from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

Conservative substitution: A substitution of one amino acid residue in a protein sequence for a different amino acid residue having similar biochemical properties. Typically, conservative substitutions have little to no impact on the activity of a resulting polypeptide. For example, ideally, a dengue virus protein including one or more conservative substitutions (for example no more than 2, 5, 10, 20, 30, 40, or 50 substitutions) retains the structure and function of the wild-type protein. A polypeptide can be produced to contain one or more conservative substitutions by manipulating the nucleotide sequence that encodes that polypeptide using, for example, standard procedures such as site-directed mutagenesis or PCR. In one example, such variants can be readily selected by testing antibody cross-reactivity or its ability to induce an immune response.

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain. Examples of conservative substitutions are shown below.

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

The substitutions which in general are expected to produce the greatest changes in protein properties will be non-conservative, for instance changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

Contacting: Placement in direct physical association; includes both in solid and liquid form, which can take place either in vivo or in vitro. Contacting includes contact between one molecule and another molecule, for example the amino acid on the surface of one polypeptide, such as a peptide, that contacts another polypeptide. Contacting can also include contacting a cell for example by placing a polypeptide in direct physical association with a cell.

Control: A reference standard. In some embodiments, the control is a negative control sample obtained from a healthy patient. In other embodiments, the control is a positive control sample obtained from a patient diagnosed with a flaviviru infection. In still other embodiments, the control is a historical control or standard reference value or range of values (such as a previously tested control sample, such as a group of patients with known prognosis or outcome from an infection, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Dengue virus (DENY): An RNA virus of the family Flaviviridae, genus Flavivirus. The dengue virus genome encodes the three structural proteins (C, prM and E) that form the virus particle and seven non-structural proteins (NS1, NS2a, NS2b, NS3, NS4a, NS4b, NS5) that are only found in infected host cells, but are required for replication of the virus. There are four serotypes of dengue virus, referred to as DENY-1, DENV-2, DENV-3 and DENV-4. All four serotypes can cause the full spectrum of dengue disease. Infection with one serotype can produce lifelong immunity to that serotype. However, severe complications can occur upon subsequent infection by a different serotype. Dengue virus is primarily transmitted by Aedes mosquitoes, particularly A. aegypti. Symptoms of dengue virus infection include fever, headache, muscle and joint pain and a skin rash similar to measles. This is called dengue fever (DF). In a small percentage of cases, the infection develops into a life-threatening dengue hemorrhagic fever (DHF), typically resulting in bleeding, low platelet levels and blood plasma leakage, or into dengue shock syndrome characterized by dangerously low blood pressure.

Degenerate variant: In the context of the present disclosure, a “degenerate variant” refers to a polynucleotide encoding a peptide (DV3-D10 polypeptide) that includes a sequence that is degenerate as a result of the genetic code. There are 20 natural amino acids, most of which are specified by more than one codon. Therefore, all degenerate nucleotide sequences encoding a peptide are included as long as the amino acid sequence of the peptide encoded by the nucleotide sequence is unchanged.

Detecting: To identify the existence, presence, or fact of something. General methods of detecting are known to the skilled artisan and may be supplemented with the protocols and reagents disclosed herein. For example, included herein are methods of detecting the level of a protein in a sample or a subject.

Epitope: An antigenic determinant. These are particular chemical groups or peptide sequences on a molecule that are antigenic, such that they elicit a specific immune response, for example, an epitope is the region of an antigen to which B and/or T cells respond. An antibody binds a particular antigenic epitope, such as DV3-D10.

Expression: Transcription or translation of a nucleic acid sequence. For example, a gene is expressed when its DNA is transcribed into an RNA or RNA fragment, which in some examples is processed to become mRNA. A gene may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. The term “expression” is used herein to denote either transcription or translation. Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as μL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

Flavivirus non-structural protein: There are seven non-structural (NS) proteins of a flavivirus, NS1, NS2A, NS2B, NS3, NS4A, NS4B, and NS5, which are encoded by the portion of the flavivirus genome that is 3′ to the structural proteins. NS1 has been implicated in RNA replication and has been shown to be secreted from infected mammalian cells (Post et al., Virus Res. 18:291-302, 1991; Mackenzie et al., Virology 220:232-240, 1996; Muylaert et al., Virology 222:159-168, 1996). NS1 can elicit strong humoral immune responses and is a potential vaccine candidate (Shlesinger et al., J. Virol. 60:1153-1155, 1986; Qu et al., J. Gen. Virol. 74:89-97, 1993). NS2 is cleaved into NS2A and NS2B, with the function of NS2A remaining unknown. NS2B forms a complex with NS3 and functions as a cofactor for the NS3 protease, which cleaves portions of the virus polyprotein. NS3 also functions as an RNA helicase and is used to unwind viral RNA during replication (Li et al., J. Virol. 73:3108-3116, 1999). While the exact functions of NS4A and NS4B remain to be elucidated, they are thought to be involved in RNA replication and RNA trafficking (Lindenbach and Rice, In: Fields Virology, Knipe and Howley, eds., Lippincott, Williams, and Wilkins, 991-1041, 2001). Finally, the NS5 protein is an RNA-dependent RNA polymerase involved in genome replication (Rice et al., Science 229:726-733, 1985). NS5 also shows methyltransferase activity commonly found in RNA capping enzymes (Koonin, J. Gen. Virol. 74:733-740, 1993).

Flavivirus structural protein: The capsid (C), premembrane (prM), and envelope (E) proteins of a flavivirus are the viral structural proteins. Flavivirus genomes consist of positive-sense RNAs that are roughly 11 kb in length. The genome has a 5′ cap, but lacks a 3′ polyadenylated tail (Wengler et al., Virology 89:423-437, 1978) and is translated into one polyprotein. The structural proteins (C, prM, and E) are at the amino-terminal end of the polyprotein followed by the non-structural proteins (NS1-5). The polyprotein is cleaved by virus and host derived proteases into individual proteins. The C protein forms the viral capsid while the prM and E proteins are embedded in the surrounding envelope (Russell et al., The Togaviruses: Biology, Structure, and Replication, Schlesinger, ed., Academic Press, 1980). The E protein functions in binding to host cell receptors resulting in receptor-mediated endocytosis. In the low pH of the endosome, the E protein undergoes a conformational change causing fusion between the viral envelope and the endosomal membranes. The prM protein is believed to stabilize the E protein until the virus exits the infected cell, at which time prM is cleaved to the mature M protein (Reviewed in Lindenbach and Rice, In: Fields Virology, Knipe and Howley, eds., Lippincott, Williams, and Wilkins, 991-1041, 2001).

Fusion protein: A protein generated by expression of a nucleic acid sequence engineered from nucleic acid sequences encoding at least a portion of two different (heterologous) proteins. To create a fusion protein, the nucleic acid sequences must be in the same reading frame and contain to internal stop codons. For example, a fusion protein includes a dengue virus NS1 or DV3-D10 fused to a heterologous protein.

Heterologous: Originating from a different genetic source. A nucleic acid molecule that is heterologous to a cell originated from a genetic source other than the cell in which it is expressed. In one specific, non-limiting example, a heterologous nucleic acid molecule encoding a gp120 protein is expressed in a cell, such as a mammalian cell. Methods for introducing a heterologous nucleic acid molecule in a cell or organism are well known in the art, for example transformation with a nucleic acid, including electroporation, lipofection, particle gun acceleration, and homologous recombination.

Immune response: A response of a cell of the immune system, such as a B-cell, T-cell, macrophage or polymorphonucleocyte, to a stimulus such as an antigen or vaccine. An immune response can include any cell of the body involved in a host defense response, including for example, an epithelial cell that secretes an interferon or a cytokine. An immune response includes, but is not limited to, an innate immune response or inflammation. As used herein, a protective immune response refers to an immune response that protects a subject from infection (prevents infection or prevents the development of disease associated with infection). Methods of measuring immune responses are well known in the art and include, for example, measuring proliferation and/or activity of lymphocytes (such as B or T cells), secretion of cytokines or chemokines, inflammation, antibody production and the like.

Immunize: To render a subject protected from an infectious disease, such as by vaccination.

Immunogen: A compound, composition, or substance which is capable, under appropriate conditions, of stimulating an immune response, such as the production of antibodies or a T-cell response in an animal, including compositions that are injected or absorbed into an animal. As used herein, as “immunogenic composition” is a composition comprising an immunogen (such as a flavivirus NS1 protein, such as DV3-D10).

Immunological Probe: A molecule that can be used for selection or identification of antibodies from sera which are directed against a specific epitope or epitopes, including from human patient sera.

Isolated: An “isolated” or “purified” biological component (such as a nucleic acid, peptide, protein, protein complex, or particle) has been substantially separated, produced apart from, or purified away from other biological components in the cell of the organism in which the component naturally occurs, that is, other chromosomal and extrachromosomal DNA and RNA, and proteins. Nucleic acids, peptides and proteins that have been “isolated” or “purified” thus include nucleic acids and proteins purified by standard purification methods. The term also embraces nucleic acids, peptides and proteins prepared by recombinant expression in a host cell, as well as chemically synthesized nucleic acids or proteins. The term “isolated” or “purified” does not require absolute purity; rather, it is intended as a relative term. Thus, for example, an isolated biological component is one in which the biological component is more enriched than the biological component is in its natural environment within a cell, or other production vessel. Preferably, a preparation is purified such that the biological component represents at least 50%, such as at least 70%, at least 90%, at least 95%, or greater, of the total biological component content of the preparation.

Label: A detectable compound or composition that is conjugated directly or indirectly to another molecule to facilitate detection of that molecule. Specific, non-limiting examples of labels include fluorescent tags, enzymatic linkages, and radioactive isotopes.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carrier: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compositions, such as one or more influenza vaccines, and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polynucleotide: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

“Nucleotide” includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

A first sequence is an “antisense” with respect to a second sequence if a polynucleotide whose sequence is the first sequence specifically hybridizes with a polynucleotide whose sequence is the second sequence.

Polypeptide: A polymer in which the monomers are amino acid residues which are joined together through amide bonds. When the amino acids are alpha-amino acids, either the L-optical isomer or the D-optical isomer can be used. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced. The term “residue” or “amino acid residue” includes reference to an amino acid that is incorporated into a protein, polypeptide, or peptide.

Preventing, treating or ameliorating a disease: “Preventing” a disease refers to inhibiting the full development of a disease, such as DF or DHF. “Treating” refers to a therapeutic intervention that ameliorates a sign or symptom of a disease or pathological condition after it has begun to develop. “Ameliorating” refers to the reduction in the number or severity of signs or symptoms of a disease.

Prime-boost vaccination: An immunotherapy including administration of a first immunogenic composition (the primer vaccine) followed by administration of a second immunogenic composition (the booster vaccine) to a subject to induce an immune response. The primer vaccine and/or the booster vaccine include a vector (such as a viral vector, RNA, or DNA vector) expressing the antigen to which the immune response is directed. The booster vaccine is administered to the subject after the primer vaccine; the skilled artisan will understand a suitable time interval between administration of the primer vaccine and the booster vaccine, and examples of such timeframes are disclosed herein. In some embodiments, the primer vaccine, the booster vaccine, or both primer vaccine and the booster vaccine additionally include an adjuvant.

Promoter: A promoter is an array of nucleic acid control sequences which direct transcription of a nucleic acid. A promoter includes necessary nucleic acid sequences near the start site of transcription. A promoter also optionally includes distal enhancer or repressor elements. A “constitutive promoter” is a promoter that is continuously active and is not subject to regulation by external signals or molecules. In contrast, the activity of an “inducible promoter” is regulated by an external signal or molecule (for example, a transcription factor). In some embodiments herein, the promoter is a CMV promoter.

Purified: The term “purified” does not require absolute purity; rather, it is intended as a relative term. Thus, for example, a purified peptide, protein, virus, or other active compound is one that is isolated in whole or in part from naturally associated proteins and other contaminants. In certain embodiments, the term “substantially purified” refers to a peptide, protein, virus or other active compound that has been isolated from a cell, cell culture medium, or other crude preparation and subjected to fractionation to remove various components of the initial preparation, such as proteins, cellular debris, and other components.

Recombinant: A recombinant nucleic acid, protein or virus is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, for example, by genetic engineering techniques. The term recombinant includes nucleic acids, proteins and viruses that have been altered solely by addition, substitution, or deletion of a portion of a natural nucleic acid molecule, protein or virus.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Homologs or orthologs of nucleic acid or amino acid sequences possess a relatively high degree of sequence identity/similarity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

Once aligned, the number of matches is determined by counting the number of positions where an identical nucleotide or amino acid residue is present in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a peptide sequence that has 1166 matches when aligned with a test sequence having 1554 amino acids is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of a polypeptide are typically characterized by possession of at least about 75%, for example at least about 80%, 85%, 90%, 91%, 92%, 93%, 94%, 95%, 96%, 97%, 98% or 99% sequence identity counted over the full length alignment with the amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Sambrook et al. (Molecular Cloning: A Laboratory Manual, 4^{th} ed, Cold Spring Harbor, N.Y., 2012) and Ausubel et al. (In Current Protocols in Molecular Biology, John Wiley & Sons, New York, through supplement 104, 2013). One example of a useful algorithm is PILEUP. PILEUP uses a simplification of the progressive alignment method of Feng & Doolittle, J. Mol. Evol. 35:351-360, 1987. The method used is similar to the method described by Higgins & Sharp, CABIOS 5:151-153, 1989. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395, 1984.

Another example of algorithms that are suitable for determining percent sequence identity and sequence similarity are the BLAST and the BLAST 2.0 algorithm, which are described in Altschul et al., J. Mol. Biol. 215:403-410, 1990 and Altschul et al., Nucleic Acids Res. 25:3389-3402, 1977. Software for performing BLAST analyses is publicly available through the National Center for Biotechnology Information (ncbi.nlm.nih.gov). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see Henikoff & Henikoff, Proc. Natl. Acad. Sci. USA 89:10915, 1989). An oligonucleotide is a linear polynucleotide sequence of up to about 100 nucleotide bases in length.

Another indicia of sequence similarity between two nucleic acids is the ability to hybridize. The more similar are the sequences of the two nucleic acids, the more stringent the conditions at which they will hybridize. The stringency of hybridization conditions are sequence-dependent and are different under different environmental parameters. Thus, hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ and/or M⁺⁺ concentration) of the hybridization buffer will determine the stringency of hybridization, though wash times also influence stringency. Generally, stringent conditions are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Conditions for nucleic acid hybridization and calculation of stringencies can be found, for example, in Sambrook et al., Molecular Cloning: A Laboratory Manual, Cold Spring Harbor Laboratory Press, Cold Spring Harbor, N.Y., 2001; Tijssen, Hybridization With Nucleic Acid Probes, Part I: Theory and Nucleic Acid Preparation, Laboratory Techniques in Biochemistry and Molecular Biology, Elsevier Science Ltd., NY, N.Y., 1993; and Ausubel et al. Short Protocols in Molecular Biology, 4^{th} ed., John Wiley & Sons, Inc., 1999.

Specifically bind: When referring to the formation of an antibody: antigen protein complex, refers to a binding reaction which determines the presence of a target polypeptide (for example, DV3-D10), in the presence of a heterogeneous population of proteins and other biologics. Thus, under designated conditions, an antibody binds preferentially to a particular target polypeptide (such as DV3-D10) and does not bind in a significant amount to other polypeptides, proteins or polysaccharides present in the sample or subject. Specific binding can be determined by methods known in the art. With reference to an antibody:antigen complex, specific binding of the antigen and antibody has a K_{d} of less than about 10⁻⁶ Molar, such as less than about 10⁻⁷ Molar, 10⁻⁸ Molar, 10⁻⁹, or even less than about 10⁻¹⁰ Molar.

Subject: Living multi-cellular vertebrate organisms, a category that includes both human and non-human mammals, such as non-human primates.

Therapeutically effective amount: A quantity of a specified agent sufficient to achieve a desired effect in a subject being treated with that agent. For example, this may be the amount of a dengue virus vaccine useful for eliciting an immune response in a subject and/or for preventing infection by dengue virus. Ideally, in the context of the present disclosure, a therapeutically effective amount of a flavivirus vaccine is an amount sufficient to increase resistance to, prevent, ameliorate, and/or treat infection caused by the flavivirus, such as a dengue virus, in a subject without causing a substantial cytotoxic effect in the subject. The effective amount of a flavivirus vaccine useful for increasing resistance to, preventing, ameliorating, and/or treating infection in a subject will be dependent on, for example, the subject being treated, the manner of administration of the therapeutic composition and other factors.

Transduce: A “transduced” or “transformed” cell is a cell into which has been introduced a nucleic acid molecule by molecular biology techniques. The term encompasses all techniques by which a nucleic acid molecule might be introduced into such a cell, including transfection with viral vectors, transformation with plasmid vectors, and introduction of naked DNA by electroporation, lipofection, and particle gun acceleration.

Vaccine: A preparation of immunogenic material capable of stimulating an immune response, administered for the prevention, amelioration, or treatment of infectious or other types of disease. The immunogenic material may include attenuated or killed microorganisms (such as bacteria or viruses), or antigenic proteins, peptides or DNA derived from them. An attenuated vaccine is a virulent organism that has been modified to produce a less virulent form, but nevertheless retains the ability to elicit antibodies and cell-mediated immunity against the virulent form. A killed vaccine is a previously virulent microorganism that has been killed with chemicals or heat, but elicits antibodies against the virulent microorganism. Vaccines may elicit both prophylactic (preventative) and therapeutic responses. Methods of administration vary according to the vaccine, but may include inoculation, ingestion, inhalation or other forms of administration. Vaccines may be administered with an adjuvant to boost the immune response.

Vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. An insertional vector is capable of inserting itself into a host nucleic acid. A vector can also include one or more selectable marker genes and other genetic elements. An expression vector is a vector that contains the necessary regulatory sequences to allow transcription and translation of inserted gene or genes. In some embodiments of the present disclosure, the vector encodes a NS1 protein, such as DV3-D10. In some embodiments, the vector is the pTR600 expression vector (U.S. Patent Application Publication No. 2002/0106798; Ross et al., Nat Immunol. 1(2):102-103, 2000; Green et al., Vaccine 20:242-248, 2001).

Virus-like particle (VLP): Virus particles made up of one of more viral structural proteins, but lacking the viral genome. Because VLPs lack a viral genome, they are non-infectious. In addition, VLPs can often be produced by heterologous expression and can be easily purified. Flavivirus VLPs can be produced by transfection of host cells with a plasmid encoding the prM and E proteins. After incubation of the transfected cells for an appropriate time to allow for protein expression (such as approximately 48 hours), VLPs can be isolated from cell culture supernatants.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Polypeptides, Nucleic Acid Molecules, Viral-Like Particles and Immunotherapeutic Compositions

Dengue viruses (DENV) are the most important mosquito transmitted viral pathogens infecting humans. Nearly half of the world's population lives in DENV endemic regions and millions of infections and many thousands of deaths occur annually. DENV infection produces a spectrum of disease, most commonly causing a self-limiting flu-like illness known as dengue fever, yet with increased frequency manifesting as life-threatening dengue hemorrhagic fever (DHF). DV3-D10 polypeptides, or nucleic acid molecules encoding the polypeptides, can be used, for example, in monovalent or tetravalent vaccines against flaviviruses such as dengue virus.

It is disclosed herein that a linear DV3-D10 polypeptide, such as the amino acid sequence SWKLEKASLIEVKTC (SEQ ID NO: 1), or the homologous region from another dengue virus (see below), can be used to induce an immune response to dengue virus. Thus, these DV3-D10 polypeptides, or nucleic acids encoding these DV3-D10 polypeptides, can be used to prevent or treat a dengue virus infection. These DV3-D10 do not include the full length protein. Thus, in some embodiments, the polypeptide is at most 15, 20, 25, 30, 35, 40, 45 or 50 amino acids in length. In specific non-limiting examples, the polypeptide is 15-45, 15-40, 15-35, 15-30, 15-25, or 15-20 amino acids in length.

Dengue virus DV3-D10 polypeptides are shown below:

| SEQ | |
|---|---|
| ID | |
| NO: | |
| 1 | S W K L E K A S L I E V K T C |
| 2 | T W K L A R A S F I E V K T C |
| 3 | T W K M E K A S F I E V K S C |
| 4 | T W K I E K A S F I E V K S C |
| 5 | T W K I E K A S F I E V K N C |
| 6 | S W K L E K A S F I E V K T C |
| 7 | T W Q I E K A S L I E V K T C |
| 8 | T W Q I E R A S L I E V K T C |

In this table, SEQ ID NOs: 2 and 3 are from a DV1 serotype, SEQ ID NOs: 4 and 5 are from a DV2 serotype, SEQ ID NOs: 1 and 6 are from a DV3 serotype, and SEQ ID NOs: 7 and 8 are from a DV4 serotype. The polypeptides can include, or consist of, one of SEQ ID NOs: 1-8.

These polypeptides can be used individually or in combination. Thus, in some embodiments, 2, 3, 4, 5, 6, 7 or 8 of these polypeptides can be used in combination. In other embodiments, the polypeptides are used individually.

Furthermore, polypeptides at least about 73% identical to any one of SEQ ID NOs: 1-8 are of use in the methods disclosed herein. Thus, polypeptides at least about 73%, about 75%, about 80%, about 95%, about 90%, about 95%, about 96%, about 97%, about 98%, about 99% identical to one of SEQ ID NOs: 1-8 are of use. In this context, “about” indicates within 0.03%. These polypeptides can be used individually or in combination. Thus, in some embodiments, 2, 3, 4, 5, 6, 7 or 8 of these polypeptides can be used in combination. In other embodiments, the polypeptides are used individually.

It is disclosed herein that a linear DV3-D10 polypeptide can be used to induce an immune response to a flavivirus, such as West Nile Virus (WNV), Japanese Encephalitis Virus (JEV) or Yellow Fever Virus (YFV). Thus, these DV3-D10 polypeptides, or nucleic acids encoding these DV3-D10 polypeptides, can be used to prevent or treat the respective flavivirus infection.

Flavivirus DV3-D10 polypeptides, specifically from West Nile Virus (WNV), Japanese Encephalitis Virus (JEV) and Yellow Fever (YV):

| | SEQ | |
|---|---|---|
| | ID | |
| | NO: | |
| WNV | 9 | T W K L E R A V I G E V K S C |
| JEV | 10 | T W K L E R A V F G E V K S C |
| YFV | 11 | T W M I H T L E A L D Y K E C |

In some embodiments, polypeptides at least about 73% identical to any one of SEQ ID NOs: 9-11 are of use in the methods disclosed herein. Thus, polypeptides at least about 73%, about 75%, about 80%, about 95%, about 90%, about 95%, about 96%, about 97%, about 98%, about 99% identical to one of SEQ ID NOs: 9-11 are of use. In this context, “about” indicates within 0.03%. The polypeptide can include, or consist of, one of SEQ ID NOs: 9-11.

These DV3-D10 do not include the full length protein. Thus, in some embodiments, the polypeptide is at most 15, 20, 25, 30, 35, 40, 45 or 50 amino acids in length. In specific non-limiting examples, the polypeptide is 15-45, 15-40, 15-35, 15-30, 15-25, or 15-20 amino acids in length.

In some embodiments, the methods do not include administering a full length NS1 polypeptide to the subject.

In some embodiments, additional polypeptides can be utilized with the DV3-D10 polypeptides. In some embodiments, the additional polypeptide is a structural polypeptide, such as the capsid (C), membrane (M) and envelope (E) glycoprotein or an antigen fragment thereof. In other embodiments, the additional polypeptide is a non-structural protein, such as a NS2A, NS2B, NS3, NS4A, NS4B and NS5 polypeptide or an antigenic fragment thereof. In some non-limiting examples, Domain III of the dengue envelope protein; surface envelope and/or prM (membrane) proteins from one of the four serotypes of dengue virus can be used with one or more of the disclosed DV3-D10 polypeptides.

Immunostimulatory compositions are provided herein. These immunostimulatory compositions can include, for example, an effective amount of a DV3-D10 polypeptide, a recombinant nucleic acid molecule encoding the DV3-D10 polypeptide, a VLP comprising a DV3-D10 polypeptide, or a recombinant nucleic acid molecule (such as a vector) encoding a VLP, and a pharmaceutically acceptable carrier. Optionally, the immunostimulatory composition can include an adjuvant and/or a pharmaceutically acceptable carrier.

In some embodiments, a subject is selected that has a flavivirus infection, or is at risk for a flavivirus infection. In further embodiments, the disclosed compositions are of use to treat a flavivirus infection in a subject, or to prevent a flavivius infection in a subject. The DV3-D10 polypeptides, nucleic acid molecules encoding the DV3-D10 polypeptides and VLPs disclosed herein can be used to elicit an immune response, such as a protective immune response, against a flavivirus. VLPs and methods of making VLPs are disclosed, for example, in PCT Publication No. 2010/103488 A1, which is incorporated herein by reference. Thus, these immunostimulatory compositions can be used to prevent or treat a flavivirus infection.

In some embodiments, a subject is selected that has a dengue virus infection, or is at risk for a dengue virus infection. In some embodiments, the disclosed compositions are of use to treat dengue virus infection in a subject, or prevent a flavivirus infection in a subject. Disclosed are monovalent vaccines for dengue virus (i.e. the polypeptide from single serotype of dengue virus). In other instances, the immunostimulatory compositions are multivalent (i.e. to polypeptide from more than one serotype of dengue virus) or are tetravalent vaccines for dengue virus (i.e. contain the polypeptide from all four dengue virus serotypes). The DV3-D10 polypeptides, nucleic acid molecules encoding dengue virus polypeptides, and VLPs disclosed herein can be used to elicit an immune response, such as a protective immune response, against dengue virus. Thus, these immunostimulatory compositions can be used to prevent or treat a dengue virus infection. In additional embodiments, the disclosed compositions are of use to prevent DHF in a subject with a dengue virus infection.

The provided DV3-D10 polypeptides, constructs or vectors encoding such polypeptides, can be combined with a pharmaceutically acceptable carrier or vehicle for administration to human or veterinary subjects. In a particular embodiment, the composition administered to a subject directs the synthesis of DV3-D10 polypeptide as described herein, and a cell within the body of the subject, after incorporating the nucleic acid within it, secretes VLPs comprising the DV3-D10 polypeptide. It is believed that such VLPs then serve as an in vivo immune stimulatory composition, stimulating the immune system of the subject to generate protective immunological responses. In some embodiments, more than one DV3-D10 polypeptide, construct or vector may be combined to form a single preparation.

The immunogenic formulations may be conveniently presented in unit dosage form and prepared using conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers. Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of a sterile liquid carrier, for example, water for injections, immediately prior to use. Extemporaneous injection solutions and suspensions may be prepared from sterile powders, granules and tablets commonly used by one of ordinary skill in the art.

In certain embodiments, unit dosage formulations are those containing a dose or unit, or an appropriate fraction thereof, of the administered ingredient. It should be understood that in addition to the ingredients particularly mentioned above, formulations encompassed herein may include other agents commonly used by one of ordinary skill in the art.

The compositions provided herein, including those for use as immune stimulatory compositions, may be administered through different routes, such as oral, including buccal and sublingual, rectal, parenteral, aerosol, nasal, intramuscular, subcutaneous, intradermal, and topical. They may be administered in different forms, including but not limited to solutions, emulsions and suspensions, microspheres, particles, microparticles, nanoparticles, and liposomes.

The volume of administration will vary depending on the route of administration. By way of example, intramuscular injections may range from about 0.1 ml to about 1.0 ml. Those of ordinary skill in the art will know appropriate volumes for different routes of administration.

Immune stimulatory compounds can be administered by directly injecting nucleic acid molecules encoding DV3-D10 polypeptides (broadly described in Janeway & Travers, Immunobiology: The Immune System In Health and Disease, page 13.25, Garland Publishing, Inc., New York, 1997; and McDonnell & Askari, N. Engl. J. Med. 334:42-45, 1996). Vectors that include nucleic acid molecules described herein, or that include a nucleic acid sequence encoding a DV3-D10 polypeptide may be utilized in such DNA vaccination methods.

Compositions of use include nucleic acid vaccines in which a nucleic acid molecule encoding a DV3-D10 polypeptide is administered to a subject in a pharmaceutical composition. For genetic immunization, suitable delivery methods known to those skilled in the art include direct injection of plasmid DNA into muscles (Wolff et al., Hum. Mol. Genet. 1:363, 1992), delivery of DNA complexed with specific protein carriers (Wu et al., J. Biol. Chem. 264:16985, 1989), co-precipitation of DNA with calcium phosphate (Benvenisty and Reshef, Proc. Natl. Acad. Sci. 83:9551, 1986), encapsulation of DNA in liposomes (Kaneda et al., Science 243:375, 1989), particle bombardment (Tang et al., Nature 356:152, 1992; Eisenbraun et al., DNA Cell Biol. 12:791, 1993), and in vivo infection using cloned retroviral vectors (Seeger et al., Proc. Natl. Acad. Sci. 81:5849, 1984). Similarly, nucleic acid vaccine preparations can be administered via viral carrier.

The amount of compound in each dose of a composition is selected as an amount that induces an immunostimulatory or immunoprotective response without significant, adverse side effects. Thus, such as composition is “immune stimulatory.” Such amount will vary depending upon which specific polypeptide is employed and how it is presented, such as with an adjuvant.

The effective amount, such as a therapeutically effective amount, will depend upon the severity of the disease and/or infection and the general state of the patient's health. The effective dosage of the disclosed compositions can be provided as repeated doses within a prolonged prophylaxis or treatment regimen that will yield clinically significant results to alleviate one or more symptoms or detectable conditions associated with infection. Determination of effective dosages in this context is typically based on animal model studies followed up by human clinical trials and is guided by administration protocols that significantly reduce the occurrence or severity of targeted disease symptoms or conditions in the subject. Suitable models in this regard include, for example, murine, non-human primate, and other accepted animal model subjects known in the art. Alternatively, effective dosages can be determined using in vitro models (for example, immunologic and histopathologic assays). Using such models, only ordinary calculations and adjustments are required to determine an appropriate concentration and dose to administer a therapeutically effective amount of the composition (for example, amounts that are effective to elicit a desired immune response or alleviate one or more symptoms of a targeted disease). In alternative embodiments, an effective amount or effective dose of the composition may simply inhibit one or more symptoms correlated with a disease or condition, or increase an immune response to the virus, for either therapeutic or preventative purposes.

The actual dosage will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the composition for eliciting the desired activity or biological response in the subject. Dosage regimens can be adjusted to provide an optimum prophylactic or therapeutic response. As described above in the forgoing listing of terms, an effective amount is also one in which any toxic or detrimental side effects of the disclosed antigen and/or other biologically active agent is outweighed in clinical terms by therapeutically beneficial effects.

In other embodiments, for prophylactic and therapeutic purposes, a therapeutically effective amount of a DV3-D10 polypeptide, polynucleotide encoding a DV3-D10 polypeptide, vector, VLP, composition can be administered to the subject in a single bolus delivery, via continuous delivery (for example, continuous transdermal, mucosal or intravenous delivery) over an extended time period, or in a repeated administration protocol (for example, by an hourly, daily or weekly, repeated administration protocol).

Dosage can be varied by the attending clinician to maintain a desired concentration at a target site (for example, systemic circulation). Higher or lower concentrations can be selected based on the mode of delivery, for example, intravenous, intamuscular, subcutaneous, trans-epidermal, rectal, oral, pulmonary, or intranasal delivery. Dosage can also be adjusted based on the release rate of the administered formulation, for example, of an intrapulmonary spray versus powder, sustained release oral versus injected particulate or transdermal delivery formulations, and so forth.

In some embodiments, initial injections may range from about 1 μg to about 1 mg, with some embodiments having a range of about 10 μg to about 800 μg, and still other embodiments a range of from about 25 μg to about 500 μg. Following an initial administration of the immune stimulatory composition, subjects may receive one or several booster administrations, adequately spaced. Booster administrations may range from about 1 μg to about 1 mg, with other embodiments having a range of about 10 μg to about 750 μg, and still others a range of about 50 μg to about 500 μg. Periodic boosters at intervals of 1-5 years, for instance three years, may be desirable to maintain the desired levels of protective immunity. In other embodiments, to achieve the same serum concentration level, for example, slow-release particles with a release rate of 5 nanomolar (under standard conditions) would be administered at about twice the dosage of particles with a release rate of 10 nanomolar.

A non-limiting range for a therapeutically effective amount of a DV3-D10 polypeptide is about 0.01 mg/kg body weight to about 10 mg/kg body weight, such as about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.5 mg/kg, about 2 mg/kg, about 2.5 mg/kg, about 3 mg/kg, about 4 mg/kg, about 5 mg/kg, or about 10 mg/kg, for example 0.01 mg/kg to about 1 mg/kg body weight, about 0.05 mg/kg to about 5 mg/kg body weight, about 0.2 mg/kg to about 2 mg/kg body weight, or about 1.0 mg/kg to about 10 mg/kg body weight. Actual methods for preparing administrable compositions will be known or apparent to those skilled in the art and are described in more detail in such publications as Remingtons Pharmaceutical Sciences, 19^{th} Ed., Mack Publishing Company, Easton, Pa., 1995.

In some embodiments, a subject receives a DNA prime vaccination, for example, a nucleic acid molecule (such as a vector) encoding a dengue virus E-glycoprotein polypeptide described herein, and subsequently receives a protein boost vaccination (such as recombinant, inactivated virus, or live attenuated virus). DNA prime-protein boost strategies for dengue virus are discussed further in section IX below.

Flavivirus virus polypeptides (such as DV3-D10 polypeptides) or VLPs (or nucleic acid molecules encoding dengue virus polypeptides or VLPs), or compositions thereof, are administered in any suitable manner, such as with pharmaceutically acceptable carriers. Pharmaceutically acceptable carriers are determined in part by the particular composition being administered, as well as by the particular method used to administer the composition. Accordingly, there is a wide variety of suitable formulations of pharmaceutical compositions of the present disclosure.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Particular methods for administering nucleic acid molecules are well known in the art. In some examples, the nucleic acid encoding the dengue virus polypeptide or VLP is administered by injection (such as intramuscular or intradermal injection) or by gene gun. One approach to administration of nucleic acids is direct immunization with plasmid DNA, such as with a mammalian expression plasmid. The nucleotide sequence encoding a DV3-D10 polypeptide can be placed under the control of a promoter to increase expression of the molecule.

Immunization by nucleic acid constructs is well known in the art and taught, for example, in U.S. Pat. No. 5,643,578 (which describes methods of immunizing vertebrates by introducing DNA encoding a desired antigen to elicit a cell-mediated or a humoral response), and U.S. Pat. No. 5,593,972 and U.S. Pat. No. 5,817,637 (which describe operably linking a nucleic acid sequence encoding an antigen to regulatory sequences enabling expression). U.S. Pat. No. 5,880,103 describes several methods of delivery of nucleic acids encoding immunogenic peptides or other antigens to an organism. The methods include liposomal delivery of the nucleic acids (or of the synthetic peptides themselves), and immune-stimulating constructs, or ISCOMS™, negatively charged cage-like structures of 30-40 nm in size formed spontaneously on mixing cholesterol and QUIL A™ (saponin). Protective immunity has been generated in a variety of experimental models of infection, including toxoplasmosis and Epstein-Barr virus-induced tumors, using ISCOMS™ as the delivery vehicle for antigens (Mowat and Donachie, Immunol. Today 12:383, 1991). Doses of antigen as low as 1 μg encapsulated in ISCOMS™ have been found to produce Class I mediated CTL responses (Takahashi et al., Nature 344:873, 1990).

In another approach to using nucleic acids for immunization, a DV3-D10 polypeptide can also be expressed by attenuated viral hosts or vectors or bacterial vectors. Recombinant vaccinia virus, adeno-associated virus (AAV), herpes virus, retrovirus, cytogmeglo virus or other viral vectors can be used to express the peptide or protein, thereby eliciting a CTL response. For example, vaccinia vectors and methods useful in immunization protocols are described in U.S. Pat. No. 4,722,848. BCG (Bacillus Calmette Guerin) provides another vector for expression of the peptides (see Stover, Nature 351:456-460, 1991).

In one embodiment, a nucleic acid encoding a DV3-D10 polypeptide is introduced directly into cells. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter. Typically, the DNA is injected into muscle, although it can also be injected directly into other sites, including tissues in proximity to metastases. Dosages for injection are usually around 0.5 μg/kg to about 50 mg/kg, and typically are about 0.005 mg/kg to about 5 mg/kg (see, e.g., U.S. Pat. No. 5,589,466).

Because VLPs lack infectious RNA and are non-replicating, dengue DNA vaccines do not suffer from the replication interference obstacles impeding the live-attenuated vaccine approaches (Laylor et al., Clin Exp Immunol 117:106-112, 2009; Petersen and Roehrig, J Infect Dis 196:1721-1723, 2007). Most importantly, unlike live viruses or the inactivated vaccines made from them, DNA vaccines can be readily manipulated and engineered to prime specific epitopes and redirect immune response away from immunodominant, pathogenic B cell and T cell epitopes. This sculpted immune memory priming can redirect subsequent vaccine boosts or natural exposure toward protective, flavivisu-specific epitopes increasing both vaccine safety and efficacy.

In one embodiment, a suitable immunization regimen includes at least two separate inoculations with one or more immunogenic compositions, with a second inoculation being administered more than about two, about three to eight, or about four, weeks following the first inoculation. A third inoculation can be administered several months after the second inoculation, and in specific embodiments, more than about five months after the first inoculation, more than about six months to about two years after the first inoculation, or about eight months to about one year after the first inoculation. Periodic inoculations beyond the third are also desirable to enhance the subject's “immune memory.” The adequacy of the vaccination parameters chosen, e.g., formulation, dose, regimen and the like, can be determined by taking aliquots of serum from the subject and assaying antibody titers during the course of the immunization program. Alternatively, the T cell populations can be monitored by conventional methods.

The prime can be administered as a single dose or multiple doses, for example two doses, three doses, four doses, five doses, six doses or more can be administered to a subject over days, weeks or months. The boost can be administered as a single dose or multiple doses, for example two to six doses, or more can be administered to a subject over a day, a week or months. Multiple boosts can also be given, such one to five, or more. Different dosages can be used in a series of sequential inoculations. For example a relatively large dose in a primary inoculation and then a boost with relatively smaller doses. The immune response against the selected antigenic surface can be generated by one or more inoculations of a subject.

The pharmaceutical or immune stimulatory compositions or methods of treatment may be administered in combination with other therapeutic treatments. For example, the compositions provided herein can be administered with an adjuvant, such as Freund incomplete adjuvant or Freund's complete adjuvant.

Optionally, one or more cytokines, such as IL-2, IL-6, IL-12, RANTES, GM-CSF, TNF-α, or IFN-γ, one or more growth factors, such as GM-CSF or G-CSF; one or more molecules such as OX-40L or 41 BBL, or combinations of these molecules, can be used as biological adjuvants (see, for example, Salgaller et al., 1998, J. Surg. Oncol. 68(2):122-38; Lotze et al., 2000, Cancer J. Sci. Am. 6(Suppl 1):S61-6; Cao et al., 1998, Stem Cells 16(Suppl 1):251-60; Kuiper et al., 2000, Adv. Exp. Med. Biol. 465:381-90). These molecules can be administered systemically (or locally) to the subject.

Aluminum has long been shown to stimulate the immune response against co-administered antigens, primarily by stimulating a TH₂ response and aluminum-based adjuvants were the first adjuvants registered for human use in the United States. Thus, the use of an aluminum adjuvant such as aluminum hydroxide, aluminum phosphate, or a mixture thereof is contemplated.

The disclosed polypeptides can also be used with a water-in-oil emulsion, for example, in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages).

### Methods of Diagnosis

In addition to the therapeutic methods provided above, any of the disclosed DV3-D10 antigens can be utilized to produce antigen specific immunodiagnostic reagents, for example, for serosurveillance. For example, in the case of the disclosed antigens, the presence of serum antibodies to a flavivirus, such as a dengue virus, is monitored, such as to detect a dengue virus infection and/or the presence of antibodies that specifically bind to the DV3-D10 polypeptides. The presence of antibodies that specifically bind to the DV3-D10 polypeptides can be used to determine disease severity.

Methods are further provided for a diagnostic assay to monitor flavivirus induced disease in a subject and/or to monitor the response of the subject to immunization with one or more of the disclosed antigens. By “flavivirus induced disease” is intended any disease caused by a flavivirus. In one embodiment, the flavivirus is dengue virus, and the disease is dengue virus fever or dengue hemorrhagic fever (DHF). The subject can be a human subject.

In some embodiments, method are provided for determining the prognosis of a flavivirus infection in a subject, comprising, measuring, in a biological sample from the subject, the amount of antibodies that specifically bind the amino acid sequence of SEQ ID NOs: 1-11, and comparing the amount of antibodies that specifically bind the flavivirus polypeptide to a control. An increase in the amount of antibodies that specifically bind the flavivirus polypeptide as compared to the control indicates a good prognosis for the subject. In specific non-limiting examples, the flavivirus is a dengue virus, and an increase in the antibodies that specifically bind the dengue virus polypeptide indicates that the subject will not develop dengue hemorrhagic fever. Such as subject could be treated at home, and not in a clinical setting (such as a hospital).

In addition, the detection of the antibodies that specifically bind a DV3-D10 polypeptide also allows monitoring the response of the subject to immunization with the disclosed antigen, or to any therapeutic agent. In some embodiments, the titer of the antibodies is determined.

In some embodiments, methods are provided for determining the effectiveness of a therapeutic agent for the treatment of a flavivirus infection in a subject. The methods include measuring, in a biological sample from the subject, the amount of antibodies that specifically bind one of SEQ ID NOs: 1-11, and comparing the amount of antibodies that specifically bind the amino acid sequence of SEQ ID NO: 1-11 to a control. An increase in the amount of antibodies that specifically bind the amino acid sequence of SEQ ID NO: 1-11 as compared to the control determines that the agent is effective for treating the subject.

The method includes contacting a disclosed polypeptide with a biological sample from the subject, and detecting binding of antibodies in the sample to the disclosed DV3-D10. The biological sample can be any biological sample from the subject, including but not limited to a blood, serum, or plasma sample. In some embodiments, the methods include contacting the biological sample with the polypeptide under conditions to form an immune complex, and quantitating the amount of the immune complex formed.

The antibody binding to a polypeptide, e.g., the immune complex, can be detected by any means known to one of skill in the art. This includes, but is not limited to, the use of labeled secondary antibodies that specifically bind the antibodies from the sample. Labels include radiolabels, enzymatic labels, and fluorescent labels. In other embodiments, a disclosed DV3-D10 polypeptide can be labeled directly and is used to isolate antibodies present in a subject or biological sample obtained from a subject.

Detection assays based on binding of a polypeptide to an antibody are well known in the art and include, for example, ELISA, Western blot, fluorescence activated cell sorting (FACS), radioimmunoassay and immunohistochemistry. As is well known to one of skill in the art, in some cases the detection assay further includes the step of contacting an antigen-antibody complex with a detection reagent, such as a labeled secondary antibody (e.g., an anti-isotype antibody, such as an anti-IgG antibody), or in the case of a sandwich ELISA, a second antibody that recognizes the same antigen as the first antibody and is labeled for detection. Secondary antibodies can also be conjugated to magnetic beads to allow for magnetic sorting. In other cases, the primary antigen is directly labeled. Directly labeled molecules can be used for a variety of detection assays, such as FACS. Labels include radiolabels, enzymatic labels, and fluorescent labels.

Noncompetitive immunoassays are assays in which antigen is directly detected and, in some instances the amount of antigen directly measured. Competitive assays are also of use. Enzyme mediated immunoassays such as immunofluorescence assays (IFA), enzyme linked immunosorbent assays (ELISA), immunoblotting (Western), and capture assays can be readily adapted to accomplish the detection of the DV3-D10 proteins.

An ELISA method can, for example, be as follows: (1) bind an antigen to a substrate; (2) contact the bound antigen with a fluid or tissue sample containing the antibodies to the virus; (3) contacting the product of step (2) with an antibody bound to a detectable moiety (e.g., a horseradish peroxidase enzyme or alkaline phosphatase enzyme) that specifically binds antibodies, such as IgG; (4) contact the produce of step (3) with the substrate for the enzyme and with a color reagent (if needed for detection); (6) observe the presence of reaction product.

Exemplary methods are disclosed below for detecting antibodies that specifically bind DV3-D10. One of skill in the art can readily design additional methods for the detection of these antibodies.

In one embodiment, the presence of antibodies in a sample is compared to a control. The control can be a standard value, or a biological sample from a subject known not to be infected with the flavivirus.

In one embodiment, a biological sample from a subject is contacted with a disclosed DV3-D10 polypeptide. The presence of antibodies in the sample that bind to the DV3-D10 polypeptide, as compared to a control, indicates that the subject has a flavivirus infection, such as a dengue virus infection.

The disclosed methods can also be used to determine the severity of a dengue virus infection. In one embodiment, the amount of antibodies, such as IgG, that specifically bind a dengue virus DV3-D10 polypeptide, is detected in a subject. The presence of an increased level of antibodies that specifically bind a dengue virus DV3-D10 polypeptide, as compared to a control, indicates that the subject is at risk for, or has, dengue virus fever (DF), but not dengue virus hemorrhagic fever (DHF). An increased level of antibodies that specifically bind a dengue virus DV3-D10 polypeptide, as compared to a control, indicates that the subject is not at risk for, or does not have dengue virus hemorrhagic fever (DHF). Thus, the disclosed methods can be used to determine which subject can be treated at home, and which subject should be treated in a clinical setting, such as a in a hospital. The control can be, for example, a subject known not to be infected with Dengue virus, or a standard value.

### Passive Immunization

Also disclosed herein is a method of passively immunizing a subject against a flavivirus. The method include administering to the subject a therapeutically effective amount of an antibody that specifically binds a polypeptide consisting of the amino acid sequence set forth as one of SEQ ID NflaOs: 1-11, thereby passively immunizing the subject against the flavivirus. The antibodies of use in the disclosed methods can be polyclonal or monoclonal. In some embodiments, the flavivirus is a Dengue virus.

Methods of making monoclonal antibodies are known in the art. Thus, one of skill in the art can readily produce monoclonal antibodies that specifically bind DV3-DV10. In contrast to polyclonal antibody preparations, which typically include different antibodies directed against different determinants (epitopes), each monoclonal antibody of a monoclonal antibody preparation is directed against a single determinant on an antigen, such as a DV3-D10 polypetpide. Thus, the modifier “monoclonal” indicates the character of the antibody as being obtained from a substantially homogeneous population of antibodies, and is not to be construed as requiring production of the antibody by any particular method. For example, the monoclonal antibodies can be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein. In some examples monoclonal antibodies are isolated from a subject. Monoclonal antibodies can have conservative amino acid substitutions which have substantially no effect on antigen binding or other immunoglobulin functions. (See, for example, Harlow & Lane, Antibodies, A Laboratory Manual, 2^{nd} ed. Cold Spring Harbor Publications, New York (2013).)

Typically, a naturally occurring immunoglobulin has heavy (H) chains and light (L) chains interconnected by disulfide bonds Immunoglobulin genes include the kappa, lambda, alpha, gamma, delta, epsilon and mu constant region genes, as well as the myriad immunoglobulin variable domain genes. There are two types of light chain, lambda (λ) and kappa (κ). There are five main heavy chain classes (or isotypes) which determine the functional activity of an antibody molecule: IgM, IgD, IgG, IgA and IgE. In one non-limiting example, IgG antibodies that specifically bind a DV3-D10 polypeptide

Non-limiting examples of antibodies of use in the methods disclosed herein include intact immunoglobulins and variants and fragments thereof known in the art that retain binding affinity for the antigen. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Antibody fragments include antigen binding fragments either produced by the modification of whole antibodies or those synthesized de novo using recombinant DNA methodologies (see, e.g., Kontermann and Dubel (Ed), Antibody Engineering, Vols. 1-2, 2^{nd} Ed., Springer Press, 2010).

A single-chain antibody (scFv) is a genetically engineered molecule containing the V_{H} and V_{L} domains of one or more antibody(ies) linked by a suitable polypeptide linker as a genetically fused single chain molecule (see, for example, Bird et al., Science, 242:423-426, 1988; Huston et al., Proc. Natl. Acad. Sci., 85:5879-5883, 1988; Ahmad et al., Clin. Dev. Immunol., 2012, doi:10.1155/2012/980250; Marbry, IDrugs, 13:543-549, 2010). The intramolecular orientation of the V_{H}-domain and the V_{L}-domain in a scFv, is typically not decisive for scFvs. Thus, scFvs with both possible arrangements (V_{H}-domain-linker domain-V_{L}-domain; V_{L}-domain-linker domain-V_{H}-domain) can be used.

A dsFv is also of use in the methods disclosed herein, wherein the V_{H} and V_{L} have been mutated to introduce a disulfide bond to stabilize the association of the chains. Diabodies also are included, which are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see, for example, Holliger et al., Proc. Natl. Acad. Sci., 90:6444-6448, 1993; Poljak et al., Structure, 2:1121-1123, 1994).

Antibodies of use also include genetically engineered forms such as chimeric antibodies (such as humanized murine antibodies) and heteroconjugate antibodies (such as bispecific antibodies). See also, Pierce Catalog and Handbook, 1994-1995 (Pierce Chemical Co., Rockford, Ill.); Kuby, J. Immunology, 3^{rd} Ed., W.H. Freeman & Co., New York, 1997.

In some embodiments, a therapeutically effective amount of antibodies that specifically bind a DV3-D10 polypeptide are administered to a subject with a flavivirus infection, such as a dengue virus infection. In other embodiments, a therapeutically effective amount of antibodies that specifically bind a DV3-D10 polypeptide are administered to a subject at risk for a flavivirus infection, such as a dengue virus infection, in order to prevent the infection or reduce symptoms of a subsequence infection. In specific, non-limiting examples, the disclosed methods are of use to prevent or treat DHF.

For passive immunization with an antibody or antigen binding fragment thereof, dosage ranges from about 0.0001 to about 100 mg/kg, and more usually about 0.01 to about 5 mg/kg, of the host body weight. For example, dosages can be about 1 mg/kg body weight or about 10 mg/kg body weight, or within the range of about 1 to about 10 mg/kg. An exemplary treatment regime entails administration daily, every other day, twice per week, once per week, or once bi-weekly The antibody can be administered on multiple occasions. In some methods, dosage is adjusted to achieve a plasma antibody concentration of 1-1000 μg/ml and in some methods 25-300 μg/ml. In some methods, two or more monoclonal antibodies with different binding specificities, for two different DV3-D10 polypeptides are administered simultaneously, in which case the dosage of each antibody administered falls within the ranges indicated.

Alternatively, an antibody that specifically binds a DV3-D10 can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies.

### Kits

Any of the disclosed DV3-D10 polypeptides can be provided as components of a kit. Optionally, such a kit includes additional components including packaging, instructions and various other reagents, such as buffers, substrates, antibodies or ligands, such as control antibodies or ligands, and detection reagents.

The kit can include a container and a label or package insert on or associated with the container. Suitable containers include, for example, bottles, vials, syringes, etc. The containers may be formed from a variety of materials such as glass or plastic. The container typically holds a composition including one or more of the disclosed DV3-D10 polypeptides, nucleic acids, or virus like particle including a disclosed a DV3-D10 polypeptide, which is effective for treating, preventing, diagnosing, monitoring a flavivirus (such as a dengue virus) infection or immune response.

In several embodiments the container may have a sterile access port (for example the container may be an intravenous solution bag or a vial having a stopper pierceable by a hypodermic injection needle). In some embodiments, label or package insert indicates that the composition is used for treating the particular condition.

The label or package insert typically will further include instructions for use of a polypeptide, or a nucleic acid or viral like particle, for example, in a method of treating or preventing a flavivirus infection. The package insert can include instructions customarily included in commercial packages of therapeutic products that contain information about the indications, usage, dosage, administration, contraindications and/or warnings concerning the use of such therapeutic products. The package insert can include instructions for methods of detection, and instructions, for example, for use of other reagents such as secondary antibodies, labels, dyes and detection reagents. The instructional materials may be written, in an electronic form (such as a computer diskette or compact disk) or may be visual (such as video files).

The kits may also include additional components to facilitate the particular application for which the kit is designed. The kits may additionally include buffers and other reagents routinely used for the practice of a particular method. Such kits and appropriate contents are well known to those of skill in the art.

The following examples are provided to illustrate certain particular features and/or embodiments. These examples should not be construed to limit the disclosure to the particular features or embodiments described.

### EXAMPLES

Dengue fever is caused by Dengue virus (DV), a member of genus Flavivirus with four antigenically distinct serotypes (DV1-DV4). Clinical outcome varies from a mild (DF) to a severe and sometimes fatal manifestation (DHF). NS1 plays a role on disease immunopathology and is one of the main targets of antibody responses during DV infection. However, the epitope determinants on DV NS1 were not previously identified. Continuous B cell epitope structures on NS1 protein were identified after DV3 infection. Human serum samples from a DV3 cohort were used to screen 76 overlapping peptides (15-mers) covering the DV3 NS1 protein sequence by ELISA. A reactive peptide (DV3-D10) was identified and its epitope structure on the native protein characterized. Antibody levels against DV3-D10 were determined throughout the course of the disease. Primary infections have constant, low levels of IgG anti DV3-D10, whereas secondary infections have a burst of antibody production at febrile phase of infection (1-5 days). Secondary DF cases have greater levels of DV3-D10 specific IgG also during febrile phase, before the vasculopathy symptoms take place in DHF patients; indicating that the presence of this antibody provides a protective factor against development of DHF. Thus, subunit vaccines and viral-like particles including DV3-D10 can be produced and used as vaccines.

### Example 1

### Study Population

This study used a total of 596 serum samples, from 210 DV3 immune patients, which were collected in different time points post onset of symptoms. Each patient had nearly 3 samples, in average, at different stages of infection. Among all patients analyzed, 38% developed mild disease (DF); whereas 54% developed a more severe clinical manifestation (47%, DFC; and 7%, DHF). For the remaining 8%, clinical diagnosis could not be determined and, thus, they were called Dengue cases (D; Table 1).

TABLE 1
Demographic profile of the population under study.

Clinical Outcome

| Demographics | DF | DFC | DHF | D |
|---|---|---|---|---|
| Patients (n) | 80 | 99 | 15 | 16 |
| Samples (n) | 225 | 288 | 33 | 50 |
| Sex (Fem/Male) | 40/40 | 43/56 | 11/4 | 8/8 |
| Age (Mean/SD) | 21/17 | 30/17 | 40/20 | 40/19 |
| Type of infection | 20/60 | 25/74 | 5/10 | 8/8 |
| (Primary/Secondary) | | | | |

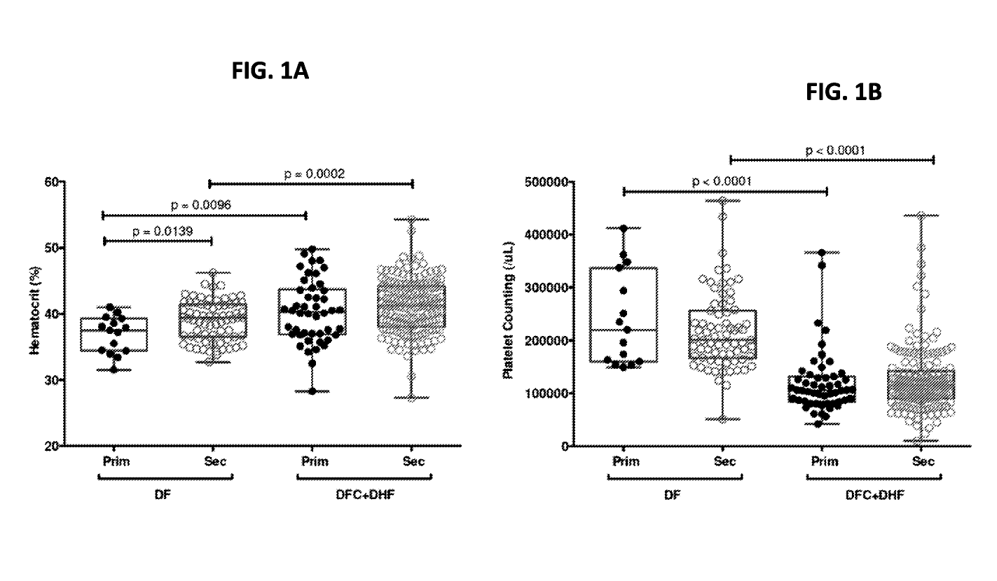

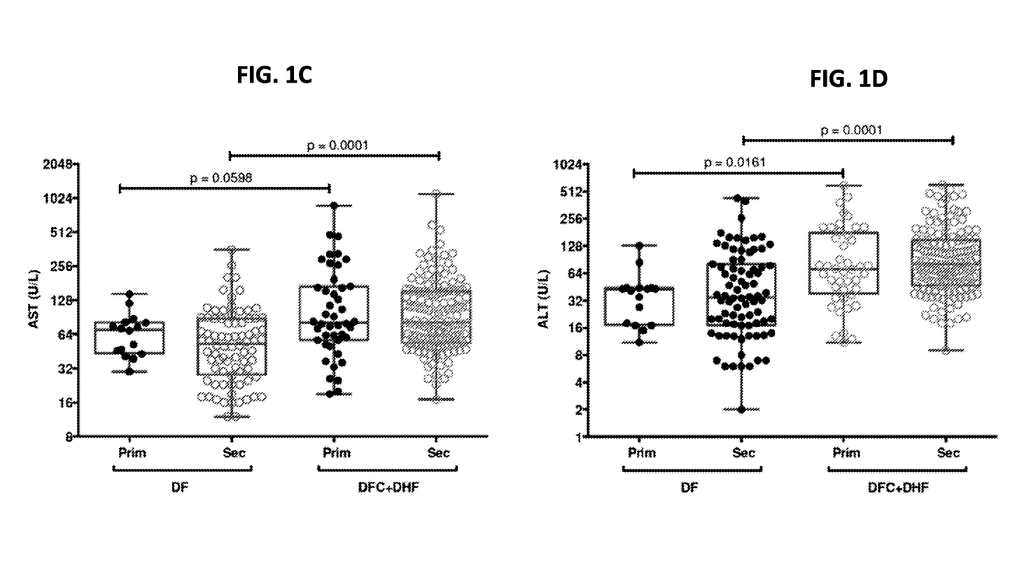

Due to the paucity of samples from DHF patients they were grouped with DFC and called severe dengue throughout the study. Blood samples from these individuals, collected up to 10 days post onset of symptoms, were used to evaluate hemoconcentration, thrombocytopenia and liver function. According to the results, individuals suffering from severe dengue, regardless of type of infection, consistently have greater hematocrit and liver enzymes (aspartate (AST) and alanine (ALT) transaminases) levels as well as lower platelet counting than mild dengue cases (FIG. 1).

### Example 2

### Identification of Linear B Cell Epitopes on DV3 NS1 Protein

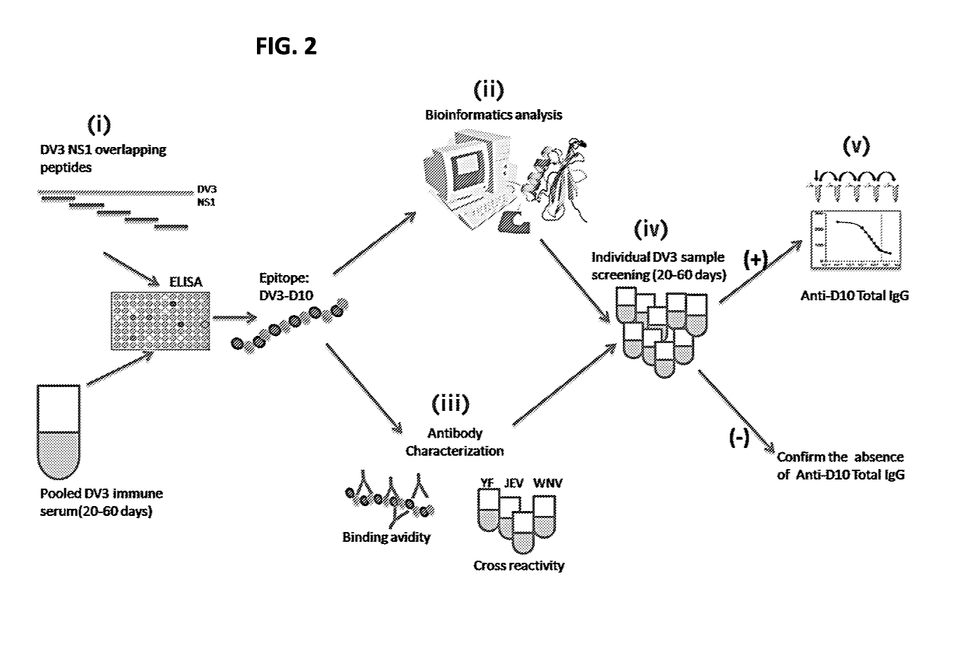

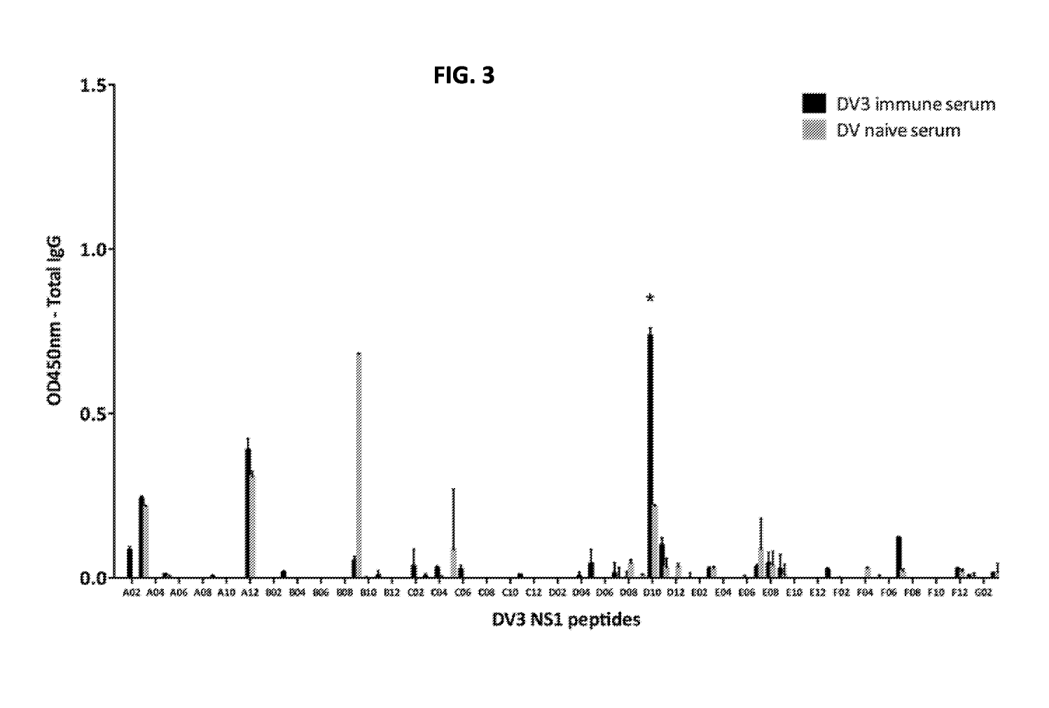

In order to map the linear B cell epitopes on DV3 NS1 protein, pooled DV3 immune sera from primary and secondary cases, collected during the pick of anti-NS1 antibody response (20-60 days after the onset of symptoms), were used to screen the DV3 NS1 peptide library (FIG. 2) for the identification of peptide-specific IgG. A peptide was considered reactive if signal on DV3 immune serum was greater than 0.2 and the ratio DV3 immune serum/DV naïve serum on the same peptide was greater than 2. Three independent screenings were performed, resulting in the identification of DV3-D10 (FIG. 3) as a potential B cell epitope. This peptide represents the amino acid sequence 209-SWKLEKASLIEVKTC-223 (SEQ ID NO: 1) on DV3 NS1 protein.

Next, the most prevalent sequences were identified, deposited on NCBI, on DV3-D10 homologous region in each DV serotype as well as other Flavivisuses (Table 2). Within DV group, DV3-D10 sequence is highly conserved on DV3 (identity above 90%), but holds an identity between 73% and 80% in relation to other DV serotypes (Table 2). DV3-D10 homologous region was even less conserved on other Flaviviruses. The sequence identities for West Nile Virus (WNV), Japanese Encephalitis Virus (JEV) and Yellow Fever Virus (YFV) were 66%, 60% and 20% respectively (Table 2).

TABLE 2
Diversity analysis of the homolgous region of
DV3-D10 on all dengue serotypes and other
Flaviviruses. Most incident protein sequences
on the DV3-D10 homologous region for DV1, DV2,
DV3, DV4, WNV and JEV were identified. These
sequences were then aligned against DV3-D10
sequence and the identity was calculated.
SEQ ID NO: 1 is shown on the top line, all
changes are relative to SEQ ID NO: 1. The
additional amino acid sequences shown in this
table are SEQ ID NOs: 2-11.

| | Inci- | | Iden- |
|---|---|---|---|
| Flavi- | dence | | tity |
| viruses | (%) | Sequence | (%) |
| DV | | S W K L E K A S L I E V K T C | |
| DV1 | 98.0 | T * * * A R * * F * * * * * * | 73.3 |
| DV2 | 46.5 | T * * M * * * * F * * * * S * | 73.3 |
| | 27.5 | T * * I * * * * F * * * * S * | 73.3 |
| | 20.5 | T * * I * * * * F * * * * N * | 73.3 |
| DV3 | 79.5 | * * * * * * * * * * * * * * * | 100.0 |
| | 19.0 | * * * * * * * * F * * * * * * | 93.3 |
| DV4 | 64.0 | T * Q I * * * * * * * * * * * | 80.0 |
| | 35.5 | T * Q I * R * * * * * * * * * | 73.3 |
| WNV | 99.7 | T * * * * R * V * G * * * S * | 66.7 |
| JEV | 90.3 | T * * * * R * V F G * * * S * | 60.0 |
| YFV | ND | T * M I H T L E A L D Y * E * | 20.0 |

### Example 3

### Cross-Reactivity with IgG Produced in Response to NS1 from Other Flaviviruses

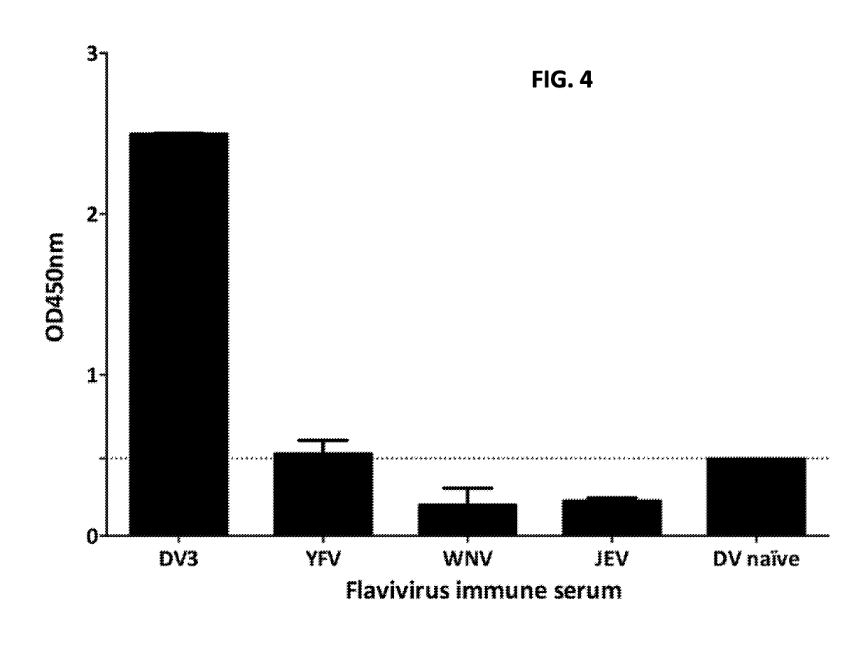

Antibodies against structural proteins (e.g. envelope) often cross-react among DV, WNV, JEV and YFV (Falconer, J Gen Virol. 2008; 89(Pt 7):1616-21; Kimura-Kuroda et al., J Gen Virol. 1986; 67 (Pt 12)(2663-72.) although this effect is less evident on antibodies against NS1 (Hirota et al., Clin Vaccine Immunol. 2012; 19(11):1853-8; Hua et al., Virus Res. 2014; 185(103-9). Thus, the ability of anti-NS1 hyperimmune serum from individuals either exposed to WNV infection or vaccinated against YF and JEV to cross-react with DV3-D10 was evaluated. According to the results, none of the hyperimmune serum samples tested recognized DV3-D10, suggesting that only antibodies originated after dengue infection interact with this peptide (FIG. 4).

### Example 4

### Antigenicity, Hydropathicity, Surface Accessibility, Flexibility and Polarity Analysis

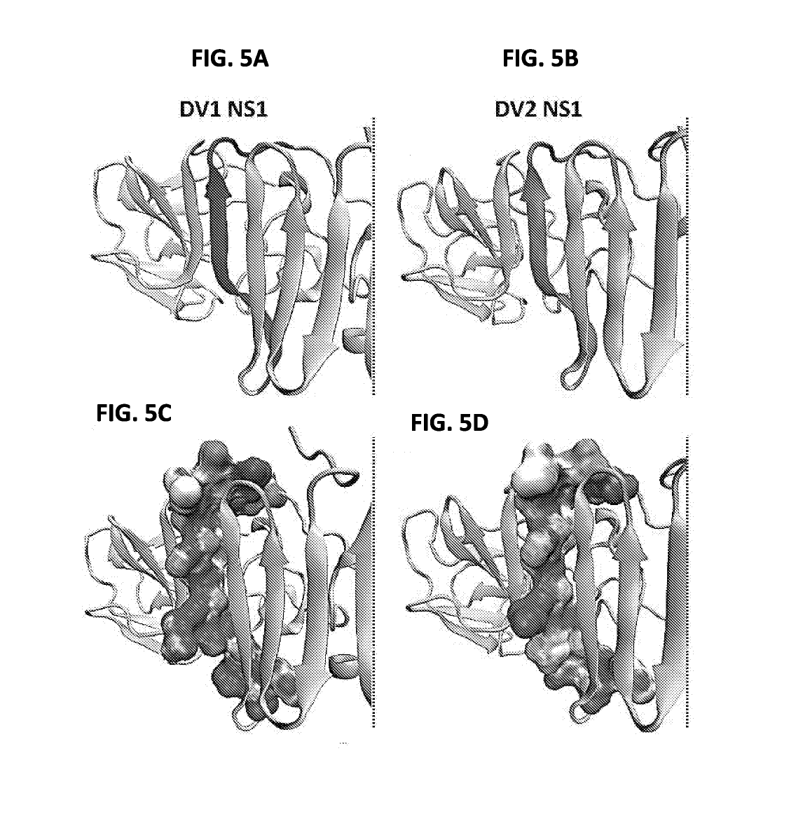

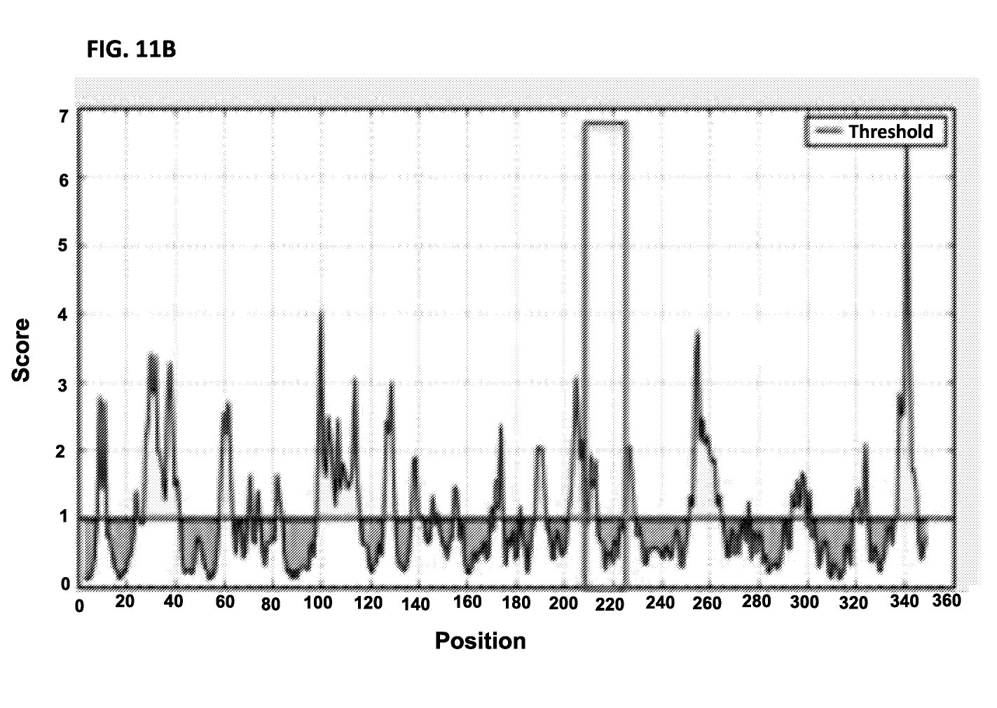

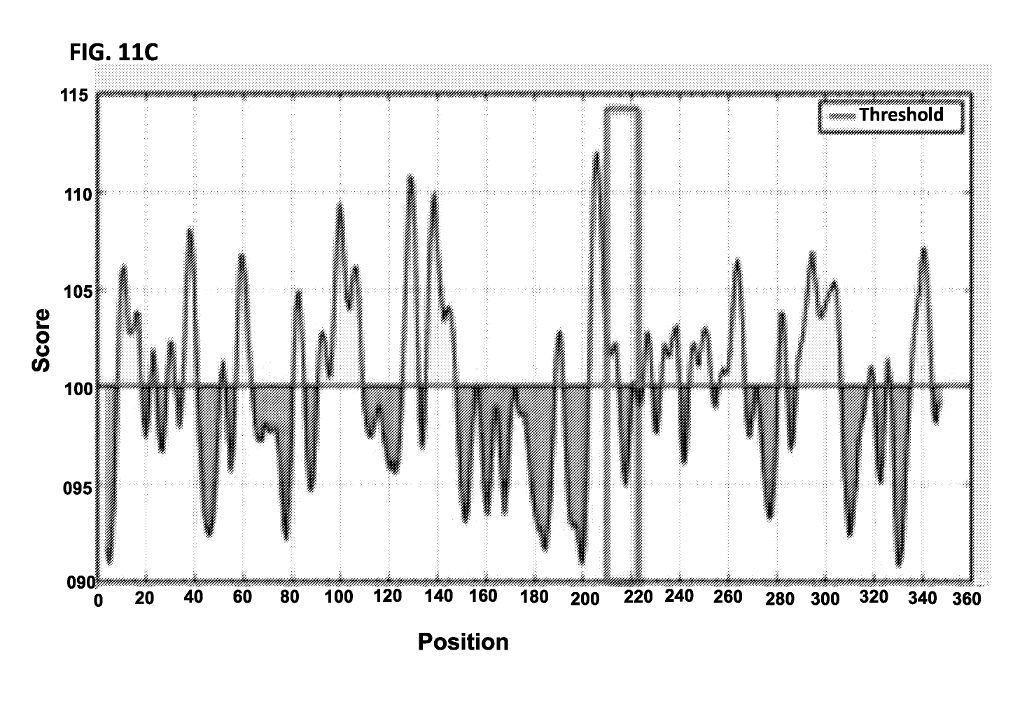

A linear B cell epitope is a continuous amino acid (aa) sequence recognized by antibodies. Hydropathicity (Kyte and Doolittle, J Mol Biol. 1982; 157(1):105-32), surface accessibility (Emini et al., J Virol. 1985; 55(3):836-9.), flexibility (Karplus and Schulz, Naturwissenschaften. 1985; 72(4):212-3) and polarity (Ponnuswamy et al., Biochim Biophys Acta. 1980; 623(2):301-16) are some of the essential features allowing the antibody to encounter and interact with a linear B cell epitope present on a folded protein. Thus, in silico analysis was carried out using the entire DV3 NS1 sequence to confirm the presence of a potential B cell epitope at the position 209-223. According to the results, DV3-D10 contains a hydrophilic, accessible and flexible region at its N-terminal portion (FIG. 11). Additionally, structural analysis using available crystal structure of the DV3-D10 homologous region on DV1 and DV2 serotypes confirmed that DV3-D10 is exposed to the surface and adopts a small loop and 43-sheet conformations (FIGS. 5A & B). The hydrophilic, accessible and flexible region at the N-terminal of the DV3-D10 contains the entire loop and only a small fraction of a β-sheet. The crystal structure also revealed that DV3-D10 had polar and charged residues facing the epitope outer surface (FIGS. 5C & D). In line with all data shown above, ABCpred server predicted, with score of 0.69, that the region 210-223 (WKLEKASLIEVKTC, amino acids 2-15 of SEQ ID NO: 1) on NS1 protein contains a potential epitope.

### Example 5

### Characterization of IgG Avidity Against DV3-D10

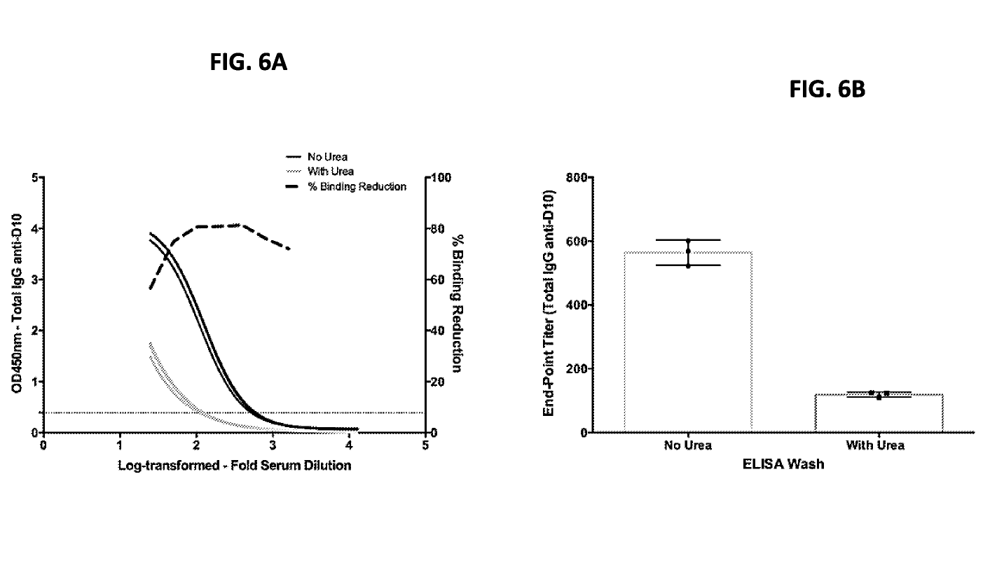

Next, the strength (IgG avidity) of the interaction between IgG and DV3-D10 was evaluated. Two-fold serially-diluted reference sample was incubated with DV3-D10 followed by a washing step with and without 6M urea (see below; (Bjorkman et al., J Vet Diagn Invest. 1999; 11(1):41-4)). According to the results, the urea wash reduced the IgG/DV3-D10 interaction by 50% to 80%, depending on the serum dilution (FIG. 6A). The end-point titer was reduced by nearly five-fold after urea wash and the calculated IgG avidity was 21% (FIG. 6B).

### Example 6

### Individual DV3 Immune Sera Screening for the Presence of IgG Anti-DV3-D10

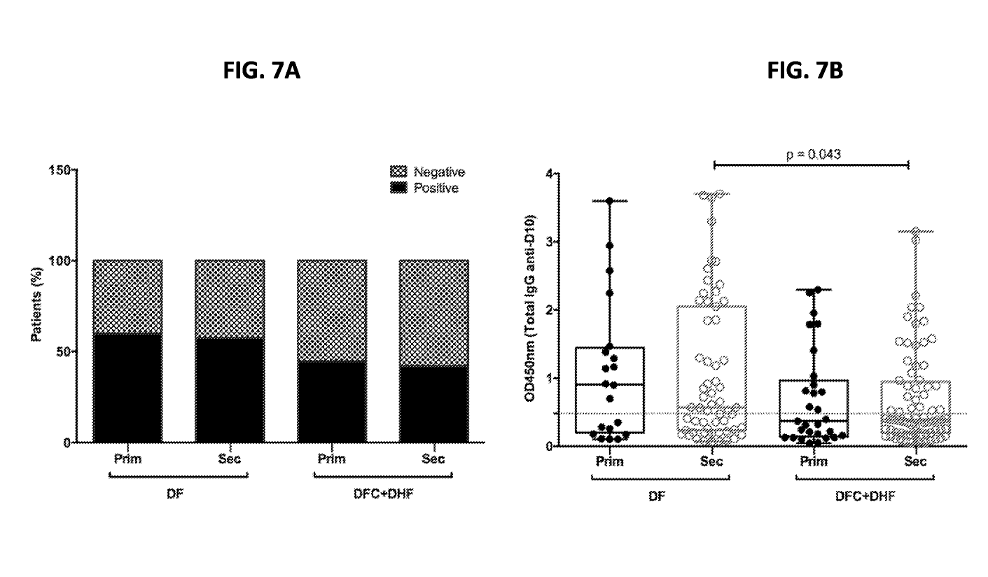

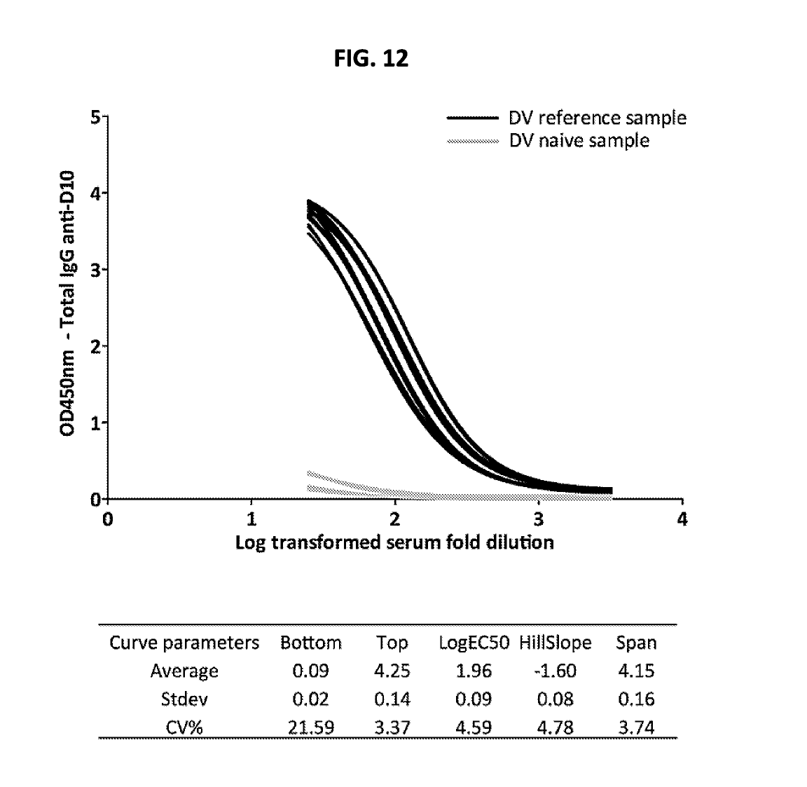

Further, a total of 200 confirmed dengue cases (57 primary and 143 secondary) were screened for the presence of IgG anti DV3-D10 using a highly reproducible and sensitive ELISA assay developed by our group (FIG. 12). Samples used on this analysis were collected between 20 and 60 days post onset of symptoms. According to the results, the overall frequency of individuals with detectable levels of IgG against this epitope was 49% (91 responders out of 184 individuals tested). The frequency of individuals positive for IgG anti-D10 was greater on mild disease (DF; 60% and 58% on primary and secondary infections respectively) as compared to a more severe syndrome (DFC+DHF; 45% and 42% on primary and secondary infections respectively; FIG. 7A). Association analysis revealed that individuals developing antibodies against DV3-D10 has nearly 2-fold more chance to develop mild disease after secondary infection (odds ratio=1.87; 95% confidence interval (0.9392-3.723); p=0.0844; Table 3). In addition, levels of response (determined per optical density—OD) were also superior on DF and restricted to secondary infections (FIG. 7B).

TABLE 3
Association analysis of presence of IgG anti DV3-D10 and occurrence of
mild disease in secondary infections.

| | | | Odds Ratio | |
|---|---|---|---|---|
| | IgG anti D10 (n) | | (95% confidence | |
| Groups | Positive | Negative | interval) | p-value* |
| DF Prim | 12 | 8 | 1.846 (0.5810-5.866) | 0.3868 |
| DFC + DHF | 13 | 16 | | |
| Prim | | | | |
| DF Sec | 34 | 25 | 1.870 (0.9392-3.723) | 0.0844 |
| DFC + DHF | 32 | 44 | | |
| Sec | | | | |

*Fisher's exact test

### Example 7

### IgG Titers Anti DV3-D10 in Confirmed Dengue Cases with Varied Clinical Outcomes

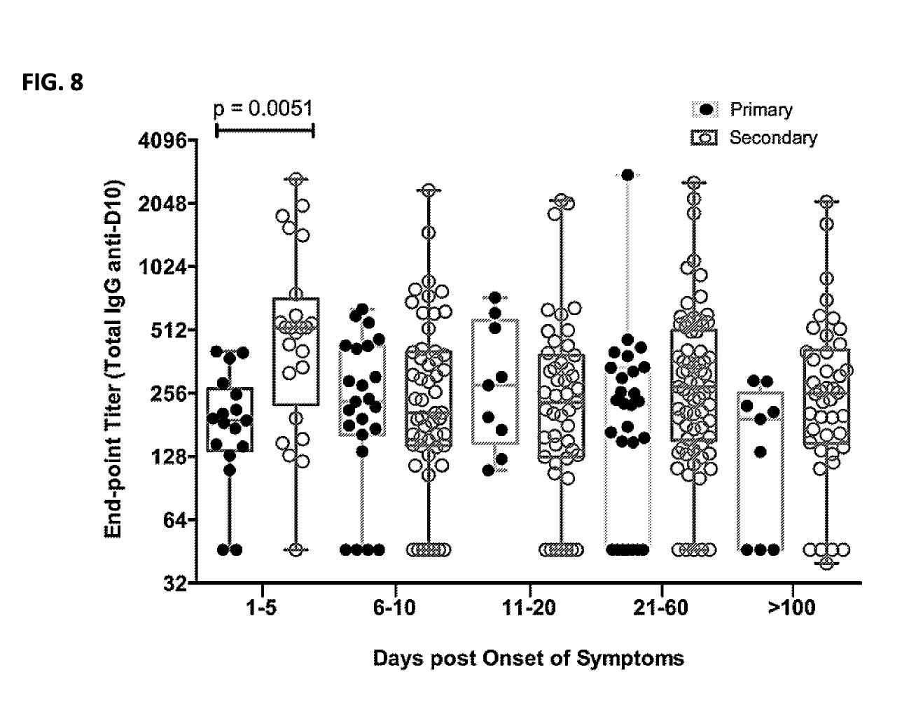

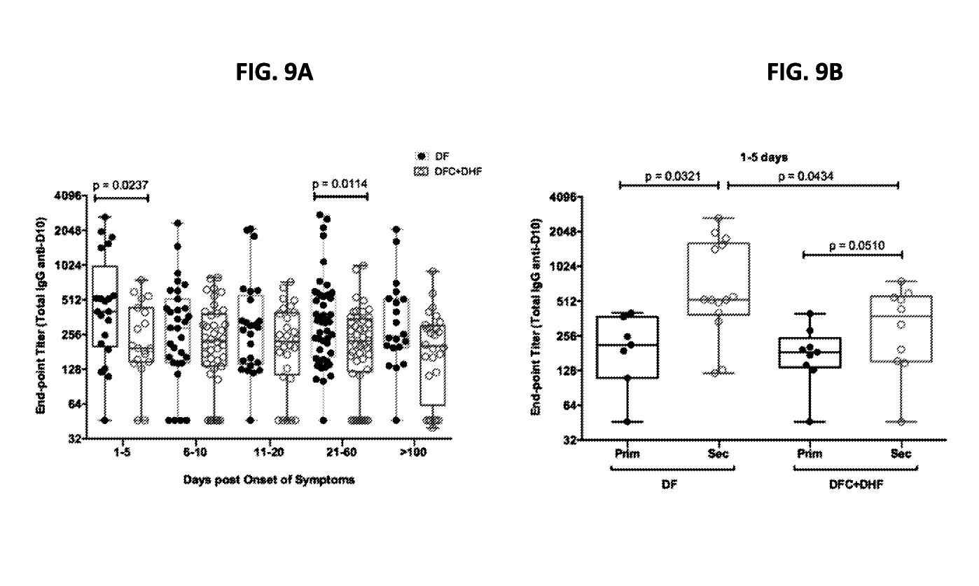

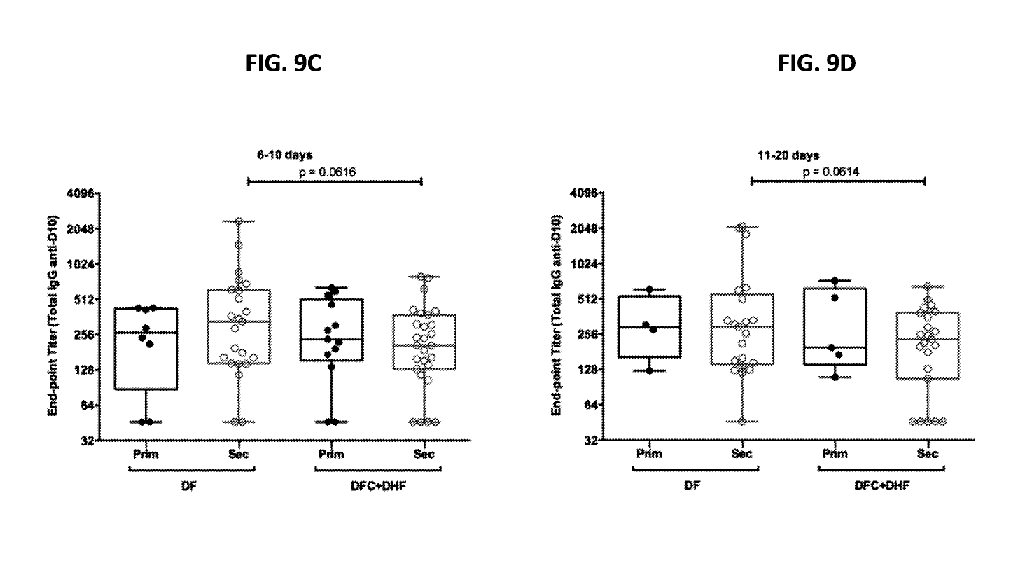

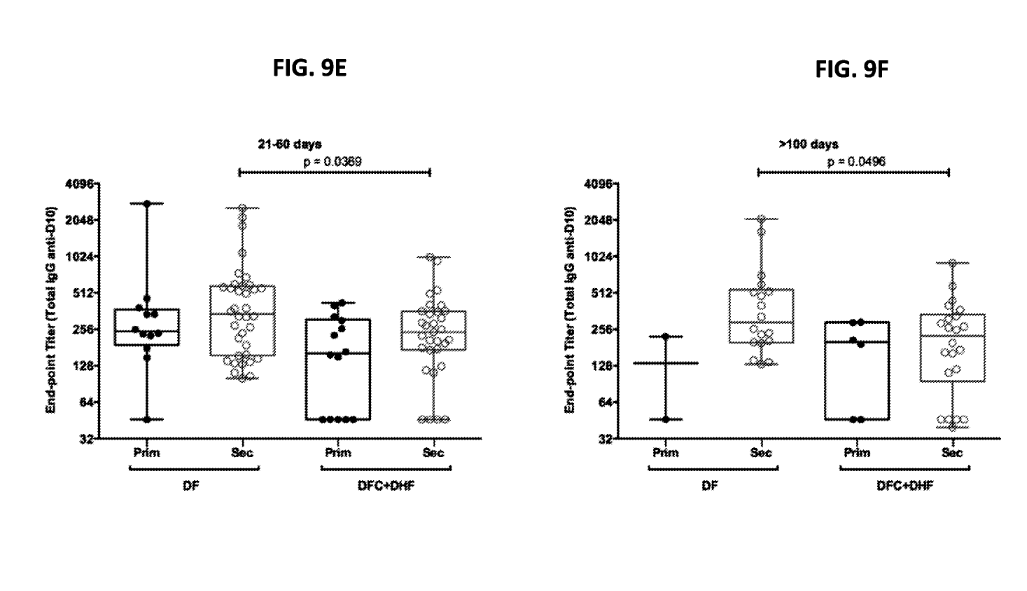

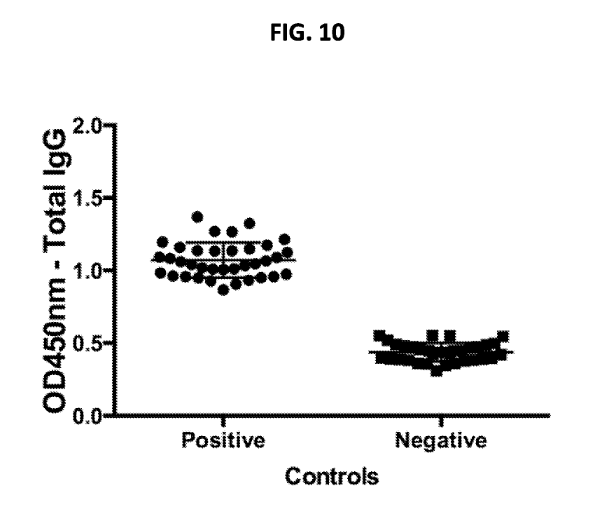

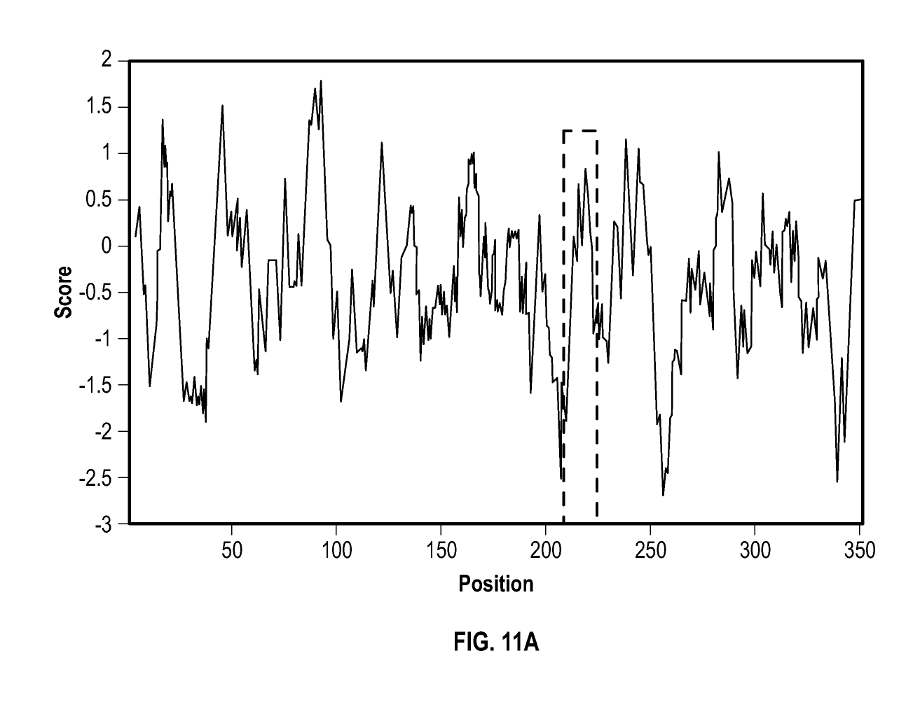

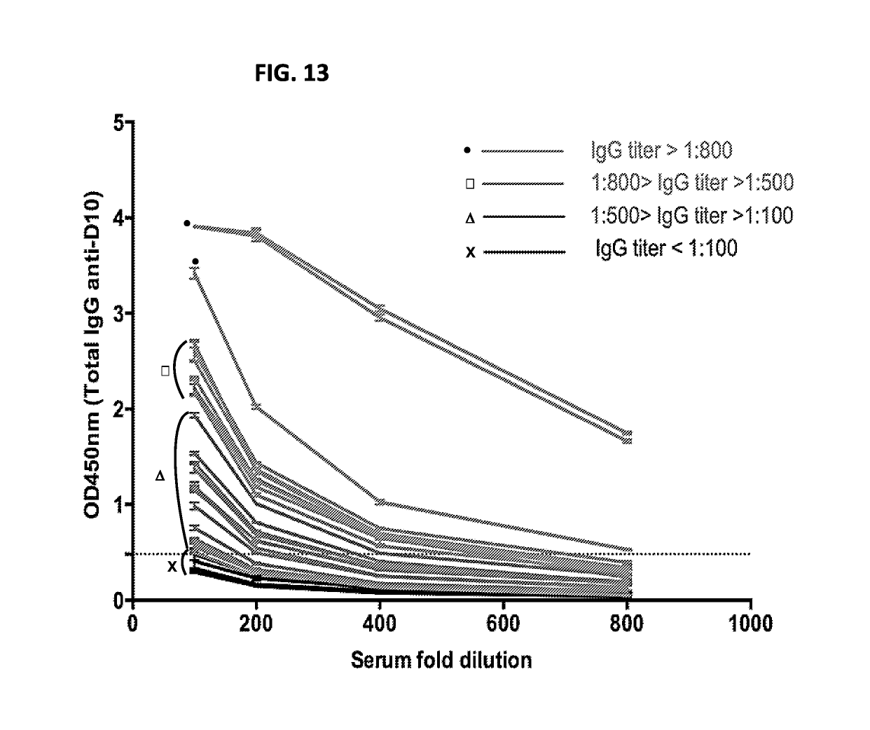

Additional serum samples were then selected from subjects positive and negative for total IgG anti DV3-D10 (FIG. 7B). For the individuals negative on the initial screening, the assay was repeated using samples collected either between 1 and 18 days or later than (>)100 days post symptoms and the absence of IgG anti DV3-D10 was confirmed. For the individuals tested positive, titers of epitope-specific IgG were determined on samples collected at different time points. Three hundred-twenty two samples from 210 patients were selected and nearly 87% of all samples analyzed had detectable levels of total IgG anti DV3-D10. A representative peptide ELISA data with different samples containing varied levels of IgG anti D10 is shown in FIG. 13. Overall, multiple virus exposures (secondary infections) increased by more than 3-fold the levels of total IgG anti-D10 at 1-5 days post symptoms (p=0.0051; FIG. 8; Table 4). Additionally, DF patients consistently produced higher antibody titers against DV3-D10 during the course of disease and after recovery as compared to DFC+DHF patients (Table 4). This difference is more evident at 1-5 days (p=0.0237) and 21-60 days (p=0.0114) post symptoms (FIG. 9A). Interestingly, IgG anti-D10 levels were greater on secondary than primary infections at only 1-5 days post symptoms (FIG. 9B) on both mild (p=0.0321) and severe (p=0.0510) diseases. Most importantly, IgG anti-D10 titers were consistently greater on DF than DFC+DHF, also on secondary infections, at 1-5 days (p=0.0434; FIG. 9B) as well as other time points analyzed (FIGS. 9C-F). Altogether, the magnitude of antibody response against this novel B cell epitope confers protection against disease severity after multiple DV exposures.

TABLE 4
Mean IgG titers anti DV3-D10, at multiple time points post onset of symptoms, on
confirmed dengue cases with varied number of virus exposures and clinical outcomes.

| | Mean IgG Titers ± SD (n) | | | | |
|---|---|---|---|---|---|
| Groups | 1-5 days | 6-10 days | 11-20 days | 21-60 days | >100 days |
| Type of | | | | | |
| Infection | | | | | |
| Primary | 205.6 ± 26.33 (17) | 275.7 ± 37.14 (23) | 339.4 ± 75.81 (9) | 308.5 ± 98.81 (27) | 164.5 ± 33.64 (9) |
| Secondary | 700.3 ± 138.7 (24) | 351.1 ± 53.67 (53) | 358.8 ± 67.28 (47) | 401.7 ± 53.22 (70) | 356.8 ± 60.42 (42) |
| Clinical | | | | | |
| Outcome | | | | | |
| DF | 227.2 ± 49.17 (7) | 264.3 ± 56.32 (8) | 331.2 ± 102.8 (4) | 470.1 ± 213.7 (12) | 134.3 ± 88.28 (2) |
| Primary | | | | | |
| DF | 933.9 ± 213.3 (14) | 477.3 ± 110.3 (23) | 515.0 ± 139.6 (21) | 519.5 ± 93.80 (36) | 500.5 ± 124.4 (18) |
| Secondary | | | | | |
| DFC + DHF | 195.4 ± 33.18 (9) | 298.9 ± 56.06 (13) | 345.9 ± 119.7 (5) | 188.7 ± 36.45 (14) | 179.5 ± 45.40 (6) |
| Primary | | | | | |
| DFC + DHF | 373.3 ± 74.43 (10) | 266.5 ± 39.15 (27) | 249.6 ± 34.64 (23) | 289.9 ± 39.96 (31) | 252.5 ± 43.82 (22) |
| Secondary | | | | | |

Thus, a novel linear B cell epitope on the region 209-223 of DV3 NS1 (here termed DV3-D10) was identified using serum samples from confirmed dengue cases collected during a period of time when only the serotype 3 was circulating in Brazil (Cordeiro et al., Am J Trop Med Hyg. 2007; 77(6):1128-3). Crystal structures on the homologous region on DV1 and DV2 confirm the accessibility of this novel epitope and denote the presence of charged residues facing its surface that influence antibody interaction. In addition, these residues form a consistent β-sheet and loop conformations on DV1 and DV2 viruses. Hydrophilicity, surface accessibility and flexibility suggest that the antibody interacts with this epitope through its loop. Moreover, epitope prediction corroborated with antigenicity analysis. This is the first time a linear B cell epitope has been identified on DV3 NS1. Other epitopes were described on DV1 and DV2, mostly using animal models immunized with recombinant protein (Chen et al., Virology. 2010; 398(2):290-8; Falconar et al., Arch Virol. 1994; 137(3-4):315-26; Jian et al., Virus Res. 2010; 150(1-2):49-5; Masrinoul et al., Jpn J Infect Dis. 2011; 64(2):109-15; Wu et al., J Clin Microbiol. 2001; 39(3):977-82; Falconar, J Clin Microbiol. 2001; 39(3):977-82), only a few of which can be recognized by serum samples from humans with history of dengue infection (Wu et al., supra; Falconar, J Clin Microbiol. 2001; 39(3):977-82). One particular epitope on DV2 NS1, termed LX2/1, described by Falconar (1997) at the position 209-TWKIEKASF-217 (SEQ ID NO: 12) partially overlaps with the homologous region on DV3-D10. The mouse monoclonal antibody (Mab) 106.3 recognizes, through an ELK/KLE-type motif, the epitope LX2/1 as well as two other regions on DV2 NS1 termed LX2/2 (267-PWHLGKLEM-275 (SEQ ID NO: 13)) and LX2/3 (331-YGMEIRPLK-339 (SEQ ID NO: 14)) (Falconar, Arch Virol. 1997; 142(5):897-916). Noteworthy, this Mab binds to human proteins also bearing the ELK/KLE-type motif, such as clotting factors and integrin/adhesion proteins (Falconar, Arch Virol. 1997; 142(5):897-916). DV3-D10 also contains an ELK/KLE-type motif (SWKLEKASLIEVKTC, SEQ ID NO: 1).

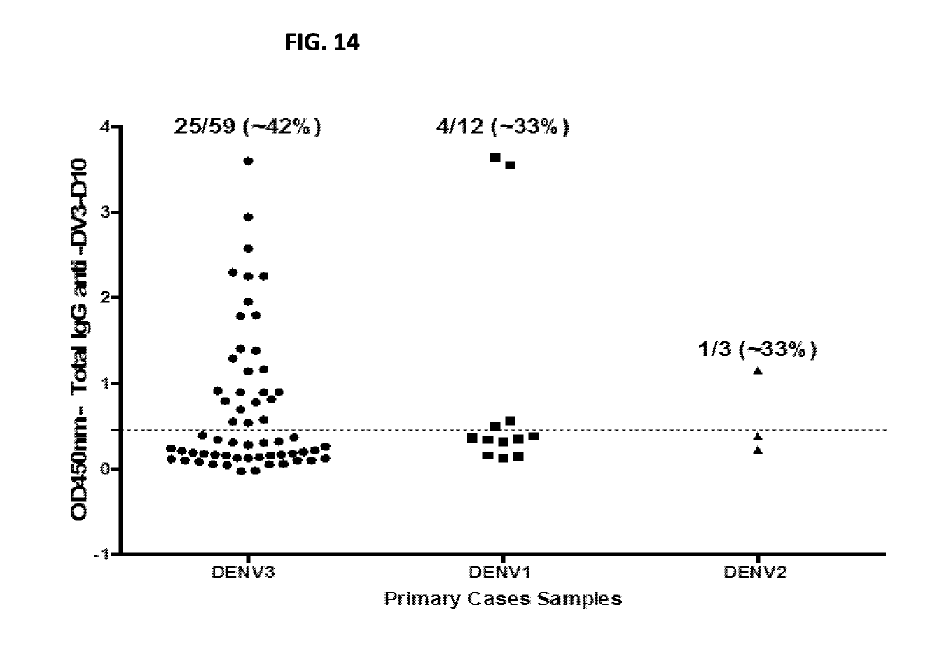

DV3-D10 sequence variability was analyzed among all DV serotypes on the most incident sequences deposited on NCBI. The identity ranged from 73% to 100% depending on the serotype (Table 2) indicating that this region is relatively conserved on the Dengue group. Thus, IgG anti DV3-D10 acquired after DV3 infection may cross-react with peptide sequences from other serotypes. Preliminary results using a limited number of dengue hyperimmune serum samples from primary DV1 and DV2 infections show the presence DV3-D10 binding IgG (FIG. 14).

Cross-reactivity analysis using serum samples containing IgG against NS1 from other Flaviviruses (WNV, JEV and YFV) allowed the determination that IgG anti DV3-D10 recognizes uniquely DV-related sequences. Lower sequence identity (20%-66%) was observed after comparing homologous DV3-D10 regions on other Flaviviruses (Table 2).

The strength of the interaction between peptide and its cognate polyclonal antibody was evaluated using a urea wash technique (Bjorkman et al., J Vet Diagn Invest. 1999; 11(1):41-4.). While the peptide/IgG bond was considered weak (IgG avidity was 21%), peptide flexibility may have influenced the immunocomplex instability, causing its dissociation upon urea treatment. Additional studies using Mab against DV3-D10 as well as the native protein are will better characterize DV3-D10/IgG interaction.

Another factor that may have influenced the interaction between IgG from dengue cases and DV3-D10 is the antigen that first activated B cells producing IgG anti DV3-D10. Several studies have suggested that antibodies recognizing DV NS1 also interact with host proteins (Cheng et al., Mol Immunol. 2009; 47(2-3):398-406; Cheng et al., Exp Biol Med (Maywood). 2009; 234(1):63-73; Lin et al., J Immunol. 2002; 169(2):657-64; Lin et al., Viral Immunol. 2006; 19(2):127-32; Liu et al., J Biol Chem. 2011; 286(11):9726-36). Thus, IgG anti DV3-D10 may have been originated by an unrelated self-antigen. The kinetics of antibody response against this epitope corroborates this hypothesis because: (i) early during the infection, IgG anti DV3-D10 is present even in primary infection; (ii) no significant increase was noted on antibody titers against DV3-D10 between primary and secondary DV infections, except at 1-5 days post onset of symptoms (FIG. 8; Table 4). Thus, IgG anti DV3-D10 may be produced in response to an unrelated self-antigen and circulate before DV infection takes place. Its levels could increase during intense inflammation at febrile phase of infection, allowing the interaction with DV NS1.

DV causes a range of clinical manifestations from asymptomatic to a mild disease called dengue fever (DF). A minority of individuals exposed to this virus experience a severe and sometime fatal syndrome called dengue hemorrhagic fever (DHF). At febrile phase of infection (around 1-5 days post symptoms), both DF and DHF patients show similar clinical picture. However, at critical phase (6-10 days), DF patients start to recover, while the clinical picture on DHF patients deteriorates.

Almost half of all patients analyzed developed antibodies against DV3-D10 (responders). This high incidence of responders may be related to the co-existence of a human T CD4+ epitope on DV3-D10 capable of binding to five HLA class II molecules highly prevalent on the population under study [DRB1*0401, DRB1*0701, DRB1*0901 and DRB3*0101 (Xavier Eurico de Alenca et al., supra; Nascimento et al., PLoS Negl Trop Dis. 2013; 7(10):e2497)]. Among the responders, DF patients with secondary infection are almost twice more likely to have IgG anti DV3-D10 (OR=1.877; p=0.08) and mount a greater antibody response (per optical density data shown on FIG. 6) to this epitope as compared to individuals developing more severe form of the disease. Further, peptide-specific IgG titers were determined on samples collected at different phases post virus infection. Primary infections were characterized by constant, low levels of IgG anti DV3-D10. In contrast, an abrupt increase on peptide-specific IgG titers was noted at febrile phase of infection (1-5 days) on secondary infections, which afterwards declined to a lower but detectable levels. Moreover, in line with initial screening, DF patients suffering from secondary infection have, consistently, greater levels of IgG anti DV3-D10 during the course of the disease, especially during febrile phase before vasculopathy symptoms take place in DHF patients; indicating that the presence of this antibody indeed represent a protective factor against development of DHF. Such effect has been observed on mice immunized with DV2 NS1, whereby immunity against NS1 was associated amelioration of virus-induced coagulopathy (Wan et al., PLoS One. 2014; 9(3):e9249). It is disclosed herein that the presence of an epitope-specific IgG against NS1 is associated with protection against DHF in humans. This antibody may interfere with direct binding of NS1 to endothelial cells, platelets or other relevant components for the virus immunopathology.

Thus, a novel B cell epitope on DV3 NS1 was identified that is highly recognized by individuals immune to DV. Antibody levels play an important role for disease outcome, since mild disease develops greater IgG response against this epitope after multiple virus exposures. DHF is proven to be a multifactorial syndrome. The results shown here suggest that higher levels of IgG specific to the novel linear B cell epitope DV3-D10 is a protective factor, on secondary infections, against DHF and shed light on the mechanism involved on the low prevalence of DHF after multiple exposure to DV. These results also provide therapeutic and prophylactic agents against vasculopathy caused by DV.

### Example 8

### Methods

Patients:

Written consent to participate in the study was obtained from each subject (or the guardian of the patient) after a full explanation of the study. All data were handled confidentially and anonymously.

Peptide Library:

A library composed of 76 synthetic peptides (15-mers), overlapping by 10-11 amino acids) was synthesized by Schafer-N (Denmark) based on the sequence of DENV3 Philippines/H87/1956 isolate (UniProtKB/Swiss-ProtAccession: P27915; GenPept: AAA99437). Both Schafer-N (Denmark) and GenScript Corporation (New Jersey, USA) synthesized the peptide DV3-D10 with purity above 80%, yielding equivalent immunoassay performances.

Serum Samples from Dengue Cases:

Longitudinal serum samples from confirmed dengue cases used in this study were obtained from a well-characterized hospital-based cohort of confirmed DV3 cases established in Recife, Brazil (Cordeiro et al., Am J Trop Med Hyg. 2007; 77(6):1128-34). Dengue diagnostics included detection of viral RNA by RT-PCR and virus isolation in C6/36 mosquito cell lines on acute samples collected during the patient hospital admission. In addition, serology for detection of virus-specific IgM and IgG, using commercial ELISA kits, on the first and follow up samples was used for diagnosis purposes as well as to determine the type of infection (whether primary or secondary (Cordeiro et al., PLoS One. 2009; 4(4):e494). Clinical diagnosis was performed based on the World Health Organization (WHO) guidelines established in 1997, and included dengue fever (DF) and dengue hemorrhagic fever (DHF). Subjects who developed severe disease (usually thrombocytopenia) but did not completely fulfilled WHO criteria for DHF were classified as complicated dengue fever (DFC). The time points assigned for each sample were determined based on the days after onset of symptoms reported by the patients during the questionnaire done at admission day. The samples analyzed included the ones obtained during early (1-5 days) and late (6-10 days) acute phases as well as early (11-20 days) and late (21-60 days) convalescence phases and after recovery (>100 days).

Hyper-Immune Serum Samples to Other Flaviviruses:

Hyper-immune serum samples against yellow fever virus (YFV) antigens used in this study were obtained from a well-characterized cohort of YF vaccine 17DD vaccinees established in Recife, Brazil, and detailed elsewhere (57). Detection of YFV-specific IgG and IgM was analyzed using in-house ELISA assays. Immunity to dengue virus was determined by detection of virus-specific IgG using ELISA. Samples were collected before and after immunization (30 days up to 5 years).

West Nile Virus (WNV) immune sera were collected in Idaho, USA, and kindly provided by Dr. William H. Hildebrand (Department of Microbiology and Immunology, University of Oklahoma Health Sciences Center, Oklahoma City, Okla., United States of America (58).

Japanese encephalitis virus (JEV) immune plasma was obtained commercially from NIBSC (United Kingdom), from dengue naïve healthy individuals immunized with JEV vaccine.

Linear B Cell Epitope Mapping Strategy:

The strategy for mapping linear B cell epitopes on NS1 peptide library consisted of (i) peptide screening by ELISA for detection of peptide-binding IgG using pooled serum samples from confirmed DV3 cases collected between 20 and 60 days post onset of symptoms; (ii) characterization of epitope structure and polarity on the NS1 protein and in silico analysis to determine epitope antigenicity, flexibility, hydrophobicity, accessibility; (iii) characterization of antibody-peptide binding strength as well as cross-reactivity with antibodies raised against other Flaviviruses; (iv) screening the selected epitope by ELISA using 200 individual serum samples collected between 20 and 60 days post symptoms from patients with varied types of infection and clinical outcomes; and finally (v) determine IgG levels against the selected epitope on 322 longitudinal samples (from acute to after recovery phases) collected from confirmed DV3 cases with varied types of infection and clinical outcomes (FIG. 2).

Detection of Peptide-Specific IgG by Enzyme-Linked Immunosorbent Assay (ELISA):

High binding, half area 96-well polystyrene plates (Corning, USA) were coated overnight at 4° C. with peptides at 5 μg/mL in carbonate/bicarbonate buffer (Pierce, Ill., USA). Plates were blocked with skimmed milk (Bio-Rad) at 5% (w/v) in PBS-T buffer [PBS with 0.1% (v/v) Tween 20; blocking buffer] for 15 minutes at room temperature (RT; 23° C. to 25° C.). Serum samples from confirmed DV3 cases were added in duplicate to the plates diluted in blocking buffer at 1:100 and allowed to incubate for 1 hour at RT. To determine the peptide-specific antibody titers, serum samples were 2-fold serially diluted (starting from 1:100) for a total of 4-point dilutions. For samples with IgG titers above 1:800, the assay was repeated with starting dilutions of 1:400. To test the cross-reactivity of DV3-D10 with other Flaviviruses, pooled dengue naïve WNV, YFV and JEV hyper-immune sera were added at 1:100 dilutions. On each plate, a negative and positive control serum samples were included for calculation of cut-off values and for assay quality control respectively. Commercially available pooled human serum sample (AB serum, MP Biomedicals) was used as negative control for the IgG anti-D10 assay, whereas samples from confirmed DV3 cases from Brazil were used as positive control. The negative control was tested and confirmed for the absence of IgG anti-dengue antigens and, thus, considered dengue naïve sample. Plates were washed 5 times with PBS-T and incubated for 1 hour at RT with horseradish peroxidase (HRP)-linked antibody anti-human IgG (JacksonImmunoresearch). After 5 washes with PBS-T, plates were incubated for 30 minutes at RT with TMB substrate (KPL, USA) and the reaction was stopped with 1N hydrochloric acid (HCl; Sigma). Optical densities at wavelength of 450 nm (OD450) were determined using SpectraMax Plus 340PC380 microplate spectrophotometer using the SoftMax Pro software version 6.4 (Molecular Devices). The results from all wells were subtracted from the blank before analysis. Cut-off values were calculated as the mean OD of negative control plus 3 times the standard deviation. Negative control values ranged from 0.309 to 0.553 [man/SD: 0.491/0.078 (n=34)]. Plates were only considered valid for analysis if positive control for peptide-specific IgG assay ranged from 0.868 to 1.369 [mean/SD: 1.072/0.122 (n=36)]. Endpoint antibody titers were calculated using 4-parameter non-linear regression on Prism version 6e (GraphPad Software Inc., La Jolla, Calif.).

ELISA Reproducibility Analysis:

To assure reproducibility of the ELISA to determine peptide-specific antibody titers, calibration curves were carried out using reference samples from the dengue cohort of confirmed DV3 cases. The assay was performed as described above with 2-fold serially diluted reference samples. The lowest dilution (1:25) was used to reach the saturation point of the assay and, thus, to determine the assay top signal. Additional dilution points were included until the signal reached the background levels (assay bottom). Curves were performed in multiple days by at least two different individuals for a total of 18 curves. Four-parameters non-linear regression was applied to each curve individually using Prism version 6e (GraphPad Software Inc., La Jolla, Calif.). Bottom, top, Log EC50, hillslope and span were noted for each curve and used to calculate the inter-assay variances [calculated as (SD/Mean)×100] for each parameter. The assay is highly reproducible, since inter-assay variances for top, LogEC50, hillslope and span were below 5% (FIG. 12).

Peptide-Specific IgG Avidity:

For binding avidity analysis, peptide-specific ELISA was carried out as described above with one modification: after reference sample incubation (diluted from 1:25 to 1:12800), PBS-T alone or supplemented with 6M urea were added to the wells and incubated for 5 minutes at RT. After that, the washing steps and incubation with HRP-conjugated antibodies were done as described above. The percentage IgG avidity anti D10 was calculated as follow: (end-titer with urea/end-titer without urea)×100 (46). Percentage of binding reduction for each sample dilution was calculated as 100−(OD with urea/OD without urea×100).

Epitope Sequence Identification and Structure Assignment:

Since the structure of the NS1 protein of the DV-3 virus is not yet available, structural data comparisons were carried out based on the solved structures of DV1 and DV2 NS1. Firstly, the amino acid sequence of the D10 peptide was aligned to the NS1 amino acid sequences of DV-1 (National Centre for Biotechnology Information—NCBI accession number: 589911261) and -2 (NCBI accession number: 586500415), all incorporated by reference herein, using the Basic Local Alignment Search Tool (BLAST) (Altschul et al., Nucleic Acids Res. 1997; 25(17):3389-402; Altschul et al., FEBS J. 2005; 272(20):5101-9) provided by NCBI to identify the closest sequence matches. Following, the Protein Data Bank (PDB) was searched for the DV-1 and -2 NS1 proteins' homologous regions and the secondary structures identified. The atomic coordinates of the DV-1 and DV-2 NS1 proteins were taken from the crystallographic structures of PDB IDs 4OIG (DV-1 NS1) (Edeling et al., Proc Natl Acad Sci USA. 2014; 111(11):4285-90) and 4O6B (DV-2 NS1) (Akey et al., Science. 2014; 343(6173):881-5) with 2.69 Å and 3.00 Å resolution, respectively. The homologous regions identified on those structures correspond to the residues from T209 to N222 in both cases. The secondary structure identification and system visualization were performed using the Visual Molecular Dynamics software (VMD) 1.8.7 (Humphrey et al., J Mol Graph. 1996; 14(1):33-8, 27-8).

Electrostatic Analysis:

Atomic coordinates of the homologous residue sequences to D10 in the DENV-1 and -2 NS1 proteins were used for the electrostatic calculations. Atomic charges and radii have been previously assigned for all atoms by the PDB2PQR 1.8 Server (Dolinsky et al., Nucleic Acids Res. 2007; 35(Web Server issue):W522-5), using the available Amber parameter set. The appropriate protonation state for each triable residue of every peptide was previously determined by the PROPKA 3.0 program (Rostkowski et al., BMC Struct Biol. 2011; 11(6)) and hydrogen atoms added accordingly. Electrostatic potentials were obtained by solving numerically the linear Poisson-Boltzmann equation and applying a finite-difference method (Davis and Mccammon, J Comput Chem. 1990; 11(3):401-9.; Nocholls et al., J Comput Chem. 1991; 12(4):435-4; Antosiewicz et al., Israel J Chem. 1994; 34(2):151-8) using the APBS (Adaptive Poisson Boltzmann Solver) program (Baker et al., Proc Natl Acad Sci USA. 2001; 98(18):10037-41.). A dielectric constant for solvent of 78.54 C2/N.m2 with solvent radius of 1.4 nm, surface tension of 0.105 N/m, and ionic strength of 0.150 M was used to describe the structures in aqueous solution. The internal dielectric constant of the solute was set to 1 C2/N.m2 (default value), varying as a function of distance reaching up to 78.4 C2/N.m2 at the peptide solvent-accessible regions. The dielectric coefficient describes the local polarizability. The functional form of this coefficient depends on the molecular shape. A low value (typically between 1 and 20) is usually assigned to the bimolecular core and higher values (ca. 80) are used to represent water at solvent accessible areas. Peptide structures and their corresponding electrostatic potentials were visualized and analyzed with the VMD 1.8.7 program.

In Silico Analysis:

Sequence of the epitope peptide was aligned to the NS1 amino acid sequences of DV3 (NCBI-accession number: P27915 [residue: 774-11251) using the NCBI BLAST (Altschul et al., 1997, supra; Altschul et al., 2005, supra) to identify the closest sequence matches. Full sequence of the DV3 NS1 protein was retrieved. Hydropathicity, flexibility and surface accessibility of the entire NS1 protein were analyzed by the method of Kyte and Doolittle (J Mol Biol. 1982; 157(1):105-32), Karplus & Schulz method (Naturwissenschaften. 1985; 72(4):212-3) and Emini method (J Virol. 1985; 55(3):836-9) respectively. ExPASy server (web.expasy.org/protscale/) was used to analyze hydrophaticity. Values below 0 represent hydrophilic regions on DV3 NS1 protein sequence. Surface accessibility plot and flexibility were analyzed using IEDB prediction resource website (tools.immuneepitope.org/bcell/) on the same protein. For both surface accessibility and flexibility, scores above 1 represent regions accessible on the protein surface and flexible respectively. Linear B cell epitope prediction analysis for the entire NS1 protein sequence was carried out using ABCpred server (imtech.res.in/raghava/abcpred/ABC_submission.html). Parameters chosen (window size=16aa and threshold=0.51) provided 65.93% accuracy, with sensitivity and specificity of 67.14% and 64.71% respectively.

Diversity Analysis of DV3-D10 and Variants:

NS1 protein sequences of each DENY serotype (as of July 2015) were collected from the NCBI Entrez Protein Database and aligned, following the method in Khan et al., PLoS Negl Trop Dis. 2008; 2(8):e272. The sequences in each serotype alignment corresponding to the DV3-D10 (SWKLEKASLIEVKTC, SEQ ID NO: 1) were extracted and analyzed for all the peptides variant to the epitope sequence by at least one amino acid difference. The incidence (% occurrence) of the individual variants in each DV serotype NS1 as well as WNV NS1, JEV NS1 and YFV NS1 alignment was determined (1717 DENV1 sequences, 1432 DENV2, 1038 DENV3, 189 DENV4, 216 JEV, 1016 WNV). Variant incidence on YFV was not calculated due to its low similarity with DV3-D10.

Statistical Analysis:

statistical analysis and plotting were carried out using prism version 6e for Macintosh. In order to ensure that the data are Gaussian-distributed, optic densities and IgG titers were log-transformed. Gaussianity of the log-tranformed data was confirmed by D'Agostino & Pearson omnibus normality test and Kolmogorov-Smirnov test. Two-tailed unpaired t-tests were then used to compare the levels of IgG anti DV3-D10 with Gaussian distribution. Groups whose distribution was not Gaussian, Mann-Whitney non-parametric test was applied. Two-tailed Fisher's exact test was applied to investigate association between presence of IgG anti DV3-D10 and development of disease severity on primary and secondary infections.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. An isolated polypeptide comprising an amino acid sequence at least 75% identical to one of SEQ ID NOs: 1-11, or a nucleic acid molecule encoding the polypeptide, or an isolated viral like particle comprising the polypeptide, wherein the polypeptide is at most 50 amino acids in length and does not comprise the amino acid sequence of a full-length flavivirus NS1 polypeptide.

2. The isolated polypeptide of claim 1, wherein the polypeptide is at most 20 amino acids in length.

3. The isolated polypeptide of claim 1, wherein the polypeptide comprises the amino acid sequence of one of SEQ ID NOs: 9-11.

4. The isolated polypeptide of claim 3, wherein the polypeptide consists of one of SEQ ID NOs: 9-11.

5. The isolated polypeptide of claim 1, wherein the polypeptide comprises an amino acid sequence at least 75% identical to one of SEQ ID NOs: 1-8.

6. The isolated polypeptide of claim 5, wherein the polypeptide comprises the amino acid sequence of one of SEQ ID NOs: 1-8.

7. The isolated polypeptide of claim 6, wherein the polypeptide consists of the amino acid sequence of one of SEQ ID NOs: 1-8.

8. A recombinant nucleic acid molecule comprising a heterologous promoter operably linked to a nucleic acid molecule encoding the polypeptide of claim 1.

9. A vector comprising the nucleic acid molecule of claim 8.

10. The isolated virus-like particle (VLP) of claim 1.

11. The isolated virus-like particle of claim 8, wherein the polypeptide consists of the amino acid sequence of one of SEQ ID NOs: 1-11.

12. An immunogenic composition comprising the polypeptide, the nucleic acid molecule, or the viral like particle of claim 1, and a pharmaceutically acceptable carrier.

13. The immunogenic composition of claim 12, further comprising an adjuvant.

14. A method for inducing an immune response to a flavivirus, comprising administering to the subject an effective amount of the immunogenic composition of claim 12, thereby inducing an immune response to a flavivirus.

15. The method of claim 14, wherein the subject has a flavivirus virus infection.

16. The method of claim 15, wherein the flavivirus is a dengue virus.

17. The method of claim 14, wherein the subject is healthy, and the method prevents an infection with the flavivirus or reduces the severity of a future flavivirus infection.

18. The method of claim 17, wherein the flavivirus is a dengue virus.

19. The method of claim 18, wherein the method prevents, or reduces the severity of, dengue virus hemorrhagic fever.

20. (canceled)

21. The method of claim 14, wherein the subject is human.

22. The method of claim 14, comprising a prime boost administration of the immunogenic composition.

23. A method for determining the effectiveness of a therapeutic agent for the treatment of a flavivirus infection in a subject, comprising
contacting a biological sample comprising antibodies from the subject treated with the therapeutic agent with a polypeptide consisting of one of SEQ ID NO: 1-11, under conditions sufficient to form an immune complex;
measuring the immune complex; and
wherein detection of the immune complex determines that the agent is effective for treating the subject.

24-25. (canceled)

26. A method for determining the prognosis of a flavivirus infection in a subject, comprising
contacting a biological sample comprising antibodies from the subject with a polypeptide consisting of one of SEQ ID NOs: 1-11 to form an immune complex; and
comparing the amount of the immune complex to a control,
wherein an increase in the immune complex as compared to the control indicates a good prognosis for the subject.

27-30. (canceled)

31. A method of passively immunizing a subject against a flavivirus, comprising administering to the subject a therapeutically effective amount of an antibody that specifically binds a polypeptide consisting of the amino acid sequence of one of SEQ ID NOs: 1-11, thereby passively immunizing the subject against the flavivirus.

32. The method of claim 11, wherein the flavivirus is a Dengue virus.


SEQUENCE LISTING (14 sequences)

SEQ ID NO: 1 - protein, 15 aa, Dengue virus
SWKLEKASLIEVKTC

SEQ ID NO: 2 - protein, 15 aa, Dengue virus
TWKLARASFIEVKTC

SEQ ID NO: 3 - protein, 15 aa, Dengue virus
TWKMEKASFIEVKSC

SEQ ID NO: 4 - protein, 15 aa, Dengue virus
TWKIEKASFIEVKSC

SEQ ID NO: 5 - protein, 15 aa, Dengue virus
TWKIEKASFIEVKNC

SEQ ID NO: 6 - protein, 15 aa, Dengue virus
SWKLEKASFIEVKTC

SEQ ID NO: 7 - protein, 15 aa, Dengue virus
TWQIEKASLIEVKTC

SEQ ID NO: 8 - protein, 15 aa, Dengue virus
TWQIERASLIEVKTC

SEQ ID NO: 9 - protein, 15 aa, West Nile virus
TWKLERAVLGEVKSD

SEQ ID NO: 10 - protein, 15 aa, Japanese encephalitis virus
TWKLERAVFGEVKSC

SEQ ID NO: 11 - protein, 15 aa, Yellow fever virus
TWMIHTLEALDYKEC

SEQ ID NO: 12 - protein, 9 aa, Dengue virus
TWKIEKASF

SEQ ID NO: 13 - protein, 9 aa, Dengue virus
PWHLGKLEM

SEQ ID NO: 14 - protein, 9 aa, Dengue virus
YGMEIRPLK